(19) United States
(12) Patent Application Publication
(10) Pub. No.: US 2024/0316112 A1
(43) Pub. Date: Sep. 26, 2024

(54) METHODS AND COMPOSITIONS FOR TREATING SPINAL CORD INJURY

(71) Applicant: Ramot at Tel-Aviv University Ltd., Tel-Aviv (IL)

(72) Inventors: Tal DVIR, Tel-Aviv (IL); Reuven EDRI, Tel-Aviv (IL); Lior WERTHEIM, Tel-Aviv (IL)

(73) Assignee: Ramot at Tel-Aviv University Ltd., Tel-Aviv (IL)

(21) Appl. No.: 18/674,990

(22) Filed: May 27, 2024

Related U.S. Application Data
(63) Continuation of application No. PCT/IL2022/051272, filed on Nov. 29, 2022.
(60) Provisional application No. 63/283,629, filed on Nov. 29, 2021.

(51) Int. Cl. A61K 35/30; A61K 35/12; A61L 31/14; C12N 5/071; C12N 5/0793

(52) U.S. Cl. CPC A61K 35/30; A61K 35/12; A61L 31/145; C12N 5/0619; C12N 5/0679; A61L 2300/64; A61L 2430/38; A61L 2430/40; C12N 2501/999; C12N 2506/45

(57) ABSTRACT

A composition is disclosed which comprises a plurality of fibrous particles fabricated from decellularized omentum, the fibrous particles being between 750 microns-3 mm in diameter, wherein the fibrous particles comprise a network of mature neurons. Uses thereof and methods of generating same are also disclosed.

### RELATED APPLICATIONS

This application is a Continuation of PCT Patent Application No. PCT/IL2022/051272 having International filing date of Nov. 29, 2022, which claims the benefit of priority under 35 USC § 119(e) of U.S. Provisional Patent Application No. 63/283,629 filed on Nov. 29, 2021. The contents of the above applications are all incorporated by reference as if fully set forth herein in their entirety.

### FIELD AND BACKGROUND OF THE INVENTION

The present invention, in some embodiments thereof, relates to compositions for treating spinal cord injury and more particularly chronic spinal cord injury.

Traumatic spinal cord injury (SCI) has an immediate and catastrophic impact on movement control and on all aspects of the patient's health and quality of life. The primary trauma injury causes direct damage, which often leads to death of cells, disruption to blood spinal cord barrier and degradation of the extracellular matrix (ECM). These processes initiate a secondary pro-inflammatory injury cascade, which leads to a progressive chronic tissue damage resulting in the formation of a glial scar. Although the healthy neural tissue surrounding the injury site contains cues that may promote tissue repair, the lack of a permissive microenvironment for cell growth in the scar tissue and the injury site, along with the absence of ECM-secreted axonal guidance molecules such as netrins and slits, result in poor intrinsic regeneration potential and permanent neural dysfunction. Furthermore, with time, the injured area expands, further challenging the ability for natural or intervened regeneration.

Aiming to rewire the injured spinal cord, researchers have suggested a variety of approaches including transplantation of different cell types or biomaterials⁵⁻⁷ into the injury site in the acute phase. The implantation of Schwann cells⁸, neural stem cells (NSCs) or neural progenitor cells (NPCs)^{9, 10}, and mesenchymal stem cells¹¹ has been investigated as potential therapy for spinal cord injury. However, two issues may jeopardize the success of such treatments, namely the host immune response to allogenic or xenogeneic cells, which may promote cell rejection, and implantation of dissociated cells, which do not organize into a functional network.

To overcome the risk of rejection, induced pluripotent stem cells (iPSCs) may be used. In this approach, somatic cells from the patient are reprogrammed to become pluripotent and then differentiated to the desired cell lineage. The most common strategy in regeneration of the injured spinal cord is not the direct application of these cells, but rather transplantation of various iPSCs-derived cell lines. Lu et al. inserted dissociated iPSCs-derived NSCs in a fibrin matrix two weeks after SCI induction. The cells were able to differentiate and interact with the host's neurons to form axons that extended through long distances of the white matter of the injured spinal cord¹³. In another work, iPSCs-derived neurospheres were injected into the spinal cord 9 days post SCI. The cells which were differentiated in vivo into the three neural lineages without forming teratomas, participated in remyelination and allowed improved locomotion¹⁴. Although such a cell source may be relevant for regenerating the injured spinal cord, injection of the cells into the injured area is not ideal. Once the dissociated cells in suspension or within biomaterial-based carriers are injected into the injury site, energy is invested to form cell-cell and cell-matrix interactions for tissue formation and differentiation, and for integration with the healthy part of the spinal cord¹⁵. As the scar tissue does not provide a supporting microenvironment for tissue assembly, massive cell death may occur. Therefore, insertion of a pre-formed 3D neuronal network into the injury site after removal of all or part the scar tissue may reduce the time required for regeneration and improve the efficacy of the treatment. However, the conditions for engineering functional 3D neural networks are still not fully known¹⁶.

Background art includes Edri et al., Advanced materials 31, 1803895 (2019); Shimojo, et al. Mol Brain 8, 79 (2015), WO2014/207744 and WO2017/103930.

### SUMMARY OF THE INVENTION

According to an aspect of the present invention there is provided a composition comprising a plurality of fibrous particles fabricated from decellularized omentum, the fibrous particles being between 750 microns-3 mm in diameter, wherein the fibrous particles comprise a network of mature neurons.

According to an aspect of the present invention there is provided a method of treating a chronic spinal cord injury of a subject comprising transplanting the composition described herein into the subject at the site of injury, at least three months following the spinal cord injury, thereby treating the spinal cord injury.

According to an aspect of the present invention there is provided an article of manufacture comprising:
(i) the composition described herein; and
(ii) a device for delivering the composition into a spinal cord of a subject.

According to an aspect of the present invention there is provided a method of generating the composition described herein, comprising:
(a) generating particles of decellularized omentum which comprise pluripotent stem cells, the particles having a diameter between 750 microns-3 mm;
(b) contacting the particles with at least one neuronal differentiation agent; and
(b) culturing the particles in the presence of the at least one neuronal differentiating agent under conditions that promote generation of a neuronal network of mature neurons in the particles, thereby generating the composition described herein.

According to embodiments of the invention, the fibrous particles are essentially spherical.

According to embodiments of the invention, the mature neurons comprise motor neurons.

According to embodiments of the invention, more than 50% of the motor neurons express Neuron-specific class III beta-tubulin (TUJ1), as measured by flow cytometry.

According to embodiments of the invention, more than 50% of the cells express Motor neuron and pancreas homeobox 1 (MNX1), as measured by flow cytometry.

According to embodiments of the invention, the fibers of the fibrous particles have an average diameter between 50-200 nm in diameter.

According to embodiments of the invention, the omentum comprises human omentum.

According to embodiments of the invention, the composition further comprises a pharmaceutically acceptable carrier.

According to embodiments of the invention, the transplanting is affected at least six months following the spinal cord injury.

According to embodiments of the invention, the method further comprises removing scar tissue at the site of injury from the subject prior to the transplanting.

According to embodiments of the invention, the composition comprises a pharmaceutically acceptable carrier at the time of transplanting and the method further comprises removing at least a portion of the carrier from the site of injury following the transplanting.

According to embodiments of the invention, the transplanting is affected using a syringe.

According to embodiments of the invention, an inner diameter of the syringe is between 1-5 mm.

According to embodiments of the invention, the device is a syringe.

According to embodiments of the invention, an inner diameter of the syringe is between 1-5 mm.

According to embodiments of the invention, the pluripotent stem cells are induced pluripotent stem cells.

According to embodiments of the invention, the induced pluripotent stem cells are reprogrammed from omental stromal cells.

According to embodiments of the invention, the neuronal differentiation agent is selected from the group consisting of a Transforming Growth Factor Beta Receptor 1 (ALK-5) inhibitor, morphogenic protein 4 (BMP4) inhibitor, retinoic acid, bone derived neurotrophic factor (BDNF), ascorbic acid and purmorphamine.

According to embodiments of the invention, the mature neurons comprise motor neurons.

According to embodiments of the invention, more than 50% of the motor neurons express Neuron-specific class III beta-tubulin (TUJ1), as measured by flow cytometry.

According to embodiments of the invention, more than 50% of the cells express Motor neuron and pancreas homeobox 1 (MNX1), as measured by flow cytometry.

According to embodiments of the invention, the generating is affected by:
(a) dispensing droplets of a mixture of solubilized, decellularized omentum and the pluripotent stem cells onto a solid surface; and
(b) subjecting the droplets to conditions that promote solidification of the droplets.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fec.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B are schematics illustrating the experiments performed according to embodiments of the present invention. A. The concept. Omental tissue is extracted from the patient. Then, cells and ECM are separated. While the cells are reprogrammed to become iPSCs, the ECM is processed into a thermo-responsive hydrogel. The iPSCs are then encapsulated within the omentum-based hydrogel, to create stem cell implants. The implants are subjected to a 30-day differentiation process, which mimics the embryonic spinal cord development. The obtained spinal cord neuron implants which are completely autologous, can then be implanted back into the patient. B. Study schematics. The differentiated spinal cord motor neuron implants were first characterized in vitro. Next, the therapeutic potential of these implants was evaluated in hemisection acute and chronic SCI models. The molecular, behavioral and anatomical aspects were investigated.

FIGS. 2A-L are photographs illustrating the generation and characterization of spinal cord implants. A. Native omentum. B. Decellularized omentum. C. Omentum-based hydrogel. D. Rheological properties of omentum-based hydrogel. E. SEM imaging of acellular hydrogel. Scale bar=2 μm. F. SEM imaging of iPSCs encapsulated within the hydrogel. Scale bar=5 μm. G. Flow cytometry analysis of undifferentiated iPSCs (TRA-1-60, SSEA-4) cultured for 3 days within the hydrogel. H. Immunofluorescence imaging of undifferentiated iPSCs cultured for 3 days within the omentum hydrogel and stained for Collagen (red) OCT4 (green) and KI67 (blue)I. Scale bar=50 μm. I. SEM imaging of differentiated implants (day 30). Scale bar=10 μm. J. Immunofluorescence of differentiated implants on day 30. Cells express a motor neuron specific marker (HB9; blue), a neuronal marker (TUJ1; green) and a dendritic marker (MAP2; red). Scale bar=50 μm. K. Immunofluorescence of differentiated implants on day 30. Cells express markers for synapses (SYP; blue), neuronal intermediate filaments (NFM; green) and dendritic (MAP2; red). Scale bar=50 μm. L. A heatmap of RNA-seq expression Z-scores computed for days 0, 20 and 30 of differentiation.

FIGS. 3A-J are photographs and graphs illustrating implant ECM content and cell function. A. A heatmap of RNA-seq expression Z-scores computed for 17 secreted ECM proteins on days 0, 20 and 30 of differentiation. B. Bar chart showing the enriched functions on day 30 of differentiation (top 500 genes), related to the 17 ECM genes in (A). C-E. Expression of ECM-associated proteins (NTN1 and SLIT1). TUJ1 is used to identify neurons. Scale bars=50 μm. C. iPSCs implants before differentiation. D. iPSCs-derived spinal cord neurons, differentiated for 30 days on MATRIGEL™. E. iPSCs-derived spinal cord implants on day 30 of differentiation. F. Rheological properties of acellular hydrogel and implants on days 0, 15, and 30 of differentiation. G. Neurite outgrowth from day-30 implant, cultured on a MATRIGEL™-coated surface for 72 h. Scale bar=50 μm. H. Neurite-branched network formed between 3 implants after 72 h. Scale bar=250 μm. I. Calcium response of implants to depolarization by KCl. J. Calcium response of implants to stimulation by glutamate.

FIGS. 4A-P illustrate the acute SCI model. A. Schematics of the study. Mice were hemisected at T10, leaving the left hindlimb paralyzed. Treatment was administered immediately after the injury was induced. Mice were kept alive for 3 months, during which scar analyses, Catwalk gait analysis and anterograde tracing were performed. Two weeks post tracing, the mice were transcardially perfused and the cords were extracted for analysis. B. Treatments administered: 3 control groups designated ‘Untreated’-animals treated with saline; ‘Cells’-animals treated with iPSCs-derived SC neurons in suspension; ‘Hydrogel’-animals treated with acellular omentum-based hydrogel; and an experimental group designated ‘Implants’-animals treated with iPSCs-derived SC neuro implants. C-G. Cellular analysis of lesion sites 7 days post treatment. C. Engraftment of implants 7 days post implantation. Prior to implantation, the cells were labeled with cytopainter (red). D. Representative image of microglia (IBA1) expression in the ‘Implants’ group. E. Quantification of IBA1 density in the different groups. F. Representative image of astrocytes (GFAP) in Implants group. G. Quantification of GFAP expression in the different groups. H-K: Cellular analysis of the lesion site 12 weeks post treatment. H. Representative image of neural stem cells (NESTIN) and neurons (TUJ1) in the ‘Implants’ group. I. Quantification of TUJ1 density in the different groups. J. Quantification of NESTIN density in the different groups. K. Representative image of ECM molecules (NTN1 and SLIT1). L. Montage of anterograde tracing of the ‘Implants’ group. Yellow arrows indicate axons that were observed caudal to lesion site. M. Quantification of axons at different distances from the lesion, as labeled by anterograde tracing. N-O. Catwalk gait analysis parameters 12 weeks post treatment. n. Degree of normal step sequence patterns-regularity index. o. Maximum pressure (left hind max intensity mean) exerted by the left hind paw. P. Weight of mice 12 weeks post treatment. All scale bars=100 μm.

FIGS. 5A-M illustrate the Chronic SCI model. A. Schematics of the study. Mice were hemisected at T10, leaving the left hindlimb paralyzed. Six weeks later, the lesion site was re-exposed, scar was resected, and treatments were administered into the cavity. Mice were kept alive for additional 8 weeks post treatment, during which MRI and behavioral studies were performed. Eight weeks post treatment, the cords were extracted for histological analysis. B. Coronal T2W MRI of spinal cord 5 weeks post initial SCI (1 week prior to scar resection). Yellow arrow indicates the complete hemisection performed on the left side of the spinal cord. C-F. Diffusion tensor imaging at 4 weeks post treatment. C. Glyph-based visualization of diffusion tensor shown on the background of axial diffusion tensor images. Blue indicates fibers in the rostral-caudal axis, red indicates right and left orientation both medial and lateral, while green indicates anterior to posterior orientation. D. Fiber tractography reconstructed in the axial plane. Red fibers are ipsilateral to the initial hemisection, and green fibers are contralateral (unharmed). Left panel of each treatment: front view; right panel: side view. E. Percentage of fibers passing through the lesion, normalized to the healthy side. F. Fraction anisotropy (FA) measurements. G-J. Quantification of protein expression at the lesion site 8 weeks post treatment: G. GFAP expression density. H. IBA1 expression density. I. TUJ1 expression density. J. Number of GAP43 positive cells. K-M. Behavioral studies performed throughout the recovery period (post treatment). K. Catwalk step sequence regularity index. L. Maximum pressure exerted by left hindlimb. M. Grid walk test: correct steps on the injured foot, out of all attempted steps. * p<0.05 was detected only between implants and untreated groups. ** p<0.05 was detected between implants and all control groups.

FIGS. 6A-B are photographs illustrating nuclei staining of native (left) and decellularized (right) omentum under the same exposure conditions. Scale bar=200 μm.

FIG. 7 is a fiber diameter histogram of omentum based hydrogel.

FIG. 8 is a table of 17 ECM genes and associated enriched functions. Purple indicates that the gene is associated with the enriched function. The bars indicate the number of ECM genes associated with a specific function.

FIG. 9 is a graph illustrating complex viscosity at different frequencies of acellular hydrogel and implants at days 0, 20 and 30 of differentiation.

FIG. 10 is a photograph illustrating cell engraftment of the injected cells group, 7 days post treatment. Cells were labeled with cytopainter (red) prior to transplantation. Nuclei appear in blue (Hoechst). Scale bar=100 μm.

FIG. 11 are representative images of immunolabeling at lesion site in acute model, 7 days post treatment. Scale bar=100 μm. Upper panel-Microglia (IBA1); Middle panel-Astrocyte (GFAP); Lower panel-Neuronal nuclei (NeuN in red) and astrocytes (GFAP in green).

FIG. 12 is a graph illustrating the number of proliferative astrocytes. Double positive staining of Ki67 proliferative marker and GFAP (astrocytes).

FIG. 13 are photographs illustrating immunostaining of neural stem cells (NESTIN) and neurons (TUJ1) at the lesion site of animals treated at the acute phase 12 weeks post implants treatment. Scale bar=100 μm.

FIG. 14 are photographs of anterograde tracing. Photomontage of anterograde tracing by TMRD. Yellow arrows indicate axons at or caudally to the lesion site. Scale bar=50 μm.

FIG. 15 illustrates glyph-based visualization of diffusion tensor imaging at 1 week following scar resection and post treatment. Glyphs are presented on the background of axial diffusion tensor images. Blue indicates fibers in the rostral-caudal axis, red indicates right and left orientation both medial and lateral, while green indicates anterior to posterior orientation.

FIG. 16 illustrates fiber tractography 4 weeks post scar resection and treatment. Fiber tractography reconstructed in the axial plane. Red fibers are ipsilateral to the initial hemisection, and green fibers are contralateral (unharmed). The fibers are shown from frontal view, with axial slices at the epicenter, rostral (+0.6 mm above lesion) and caudal (−0.6 mm below lesion).

FIG. 17 are photographs illustrating cellular content 8 weeks post treatment at the chronic phase. Immunostaining of astrocytes (GFAP), microglia (IBA1), nerve growth associated protein (GAP43) and neurons (TUJ1) at the lesion site. Scale bars=100 μm.

FIGS. 18A-B are graphs portraying results of behavioral studies 4 weeks post chronic scar resection and treatment. A. Grid walk testing the sensorimotor of the mice. B. Left hind max intensity, correlating to the pressure the mice place on the injured foot.

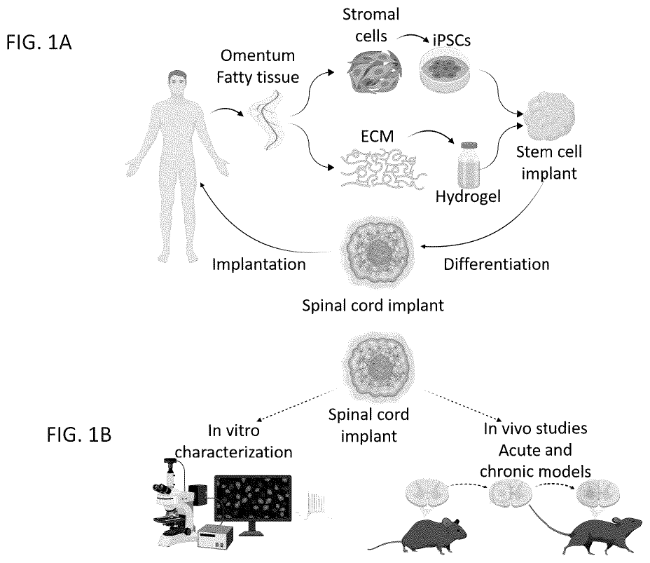

### DESCRIPTION OF SPECIFIC EMBODIMENTS OF THE INVENTION

The present invention, in some embodiments thereof, relates to compositions for treating spinal cord injury and more particularly chronic spinal cord injury.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Traumatic spinal cord injury (SCI) has an immediate and catastrophic impact on movement control and on all aspects of the patient's health and quality of life. The primary trauma injury causes direct damage, which often leads to death of cells, disruption to blood spinal cord barrier and degradation of the extracellular matrix (ECM). These processes initiate a secondary proinflammatory injury cascade, which leads to a progressive chronic tissue damage resulting in the formation of a glial scar.

The present inventors have now discovered a way to treat the injured spinal cord (SC) at the chronic stage. Using an approach which recapitulated the embryonic development of the SC, ECM-based particles (also referred to herein as mini-implants) were produced, providing an initial support and interactive material for iPSC culture and expansion. Throughout the in vitro cultivation stage, the cells and the ECM of the particles showed a synergistic effect, mimicking the process of spinal cord formation in the embryo. The ECM-based particles supported the initiation of an efficient cell differentiation in 3D by providing the cells with an adequate microenvironment. During cell differentiation, the cells continuously remodeled the fibers of the particles by secreting specific motor neuron ECM proteins, providing an inductive microenvironment for cell-cell and cell matrix interactions. Overall, this dynamic microenvironment, supplying different biochemical cues for the distinct developmental stages promoted functional SC implant assembly.

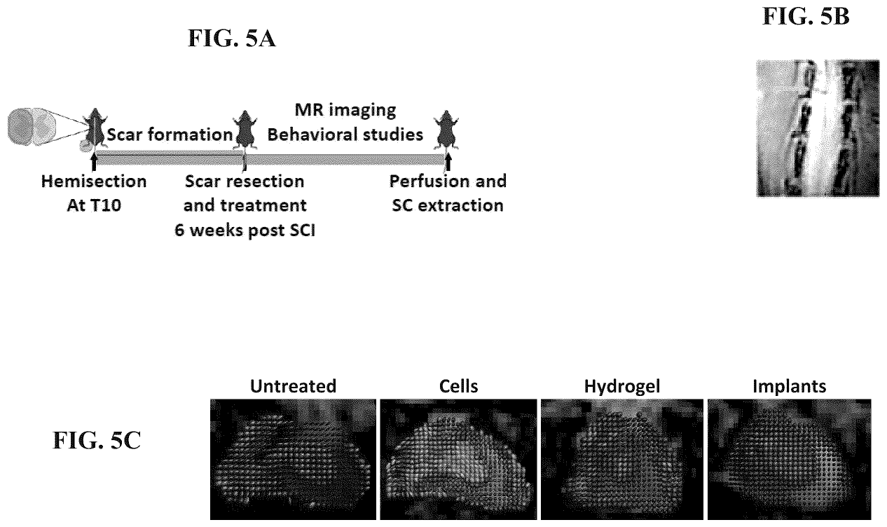

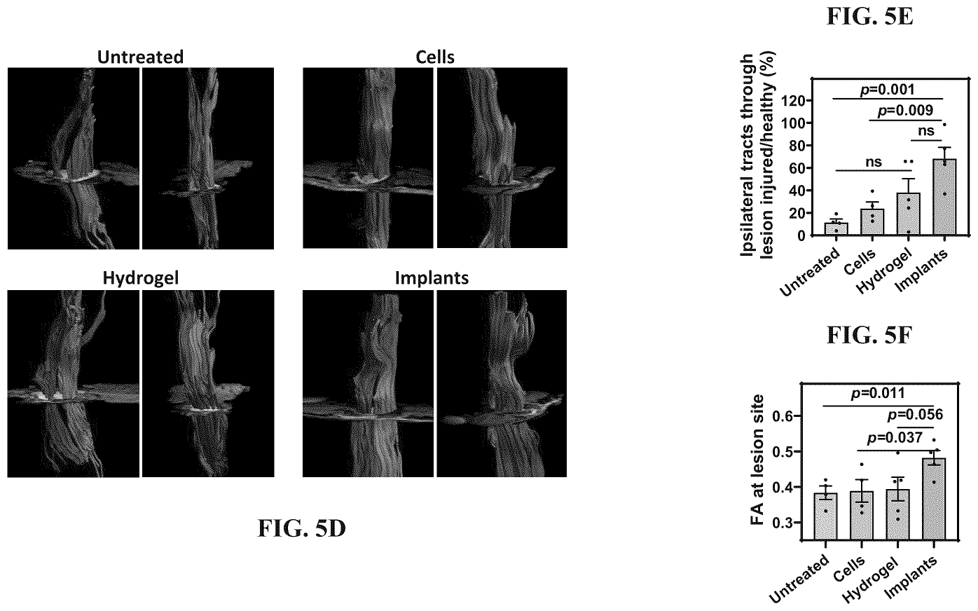

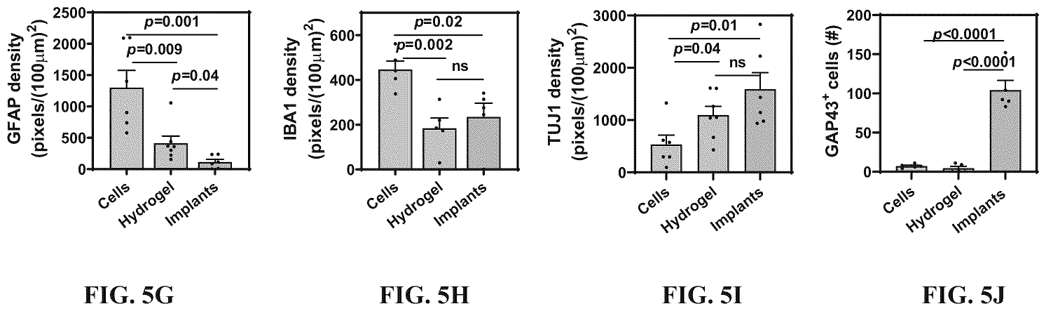

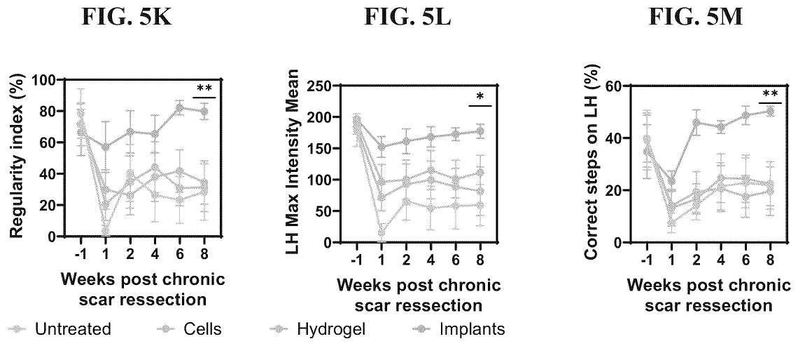

The potential of the implants (i.e. plurality of ECM-based particles) to treat the injured SC in the chronic phase was then investigated. At this stage, the scar is fully developed and spontaneous behavioral recovery has reached its plateau. After scar tissue resection and insertion of the implants so as to fill the resectioned gap formed after scar removal, the anatomy and tissue morphology were evaluated by MRI, indicating strong anisotropy and a high number of complete nerve fibers in the SC (FIG. 5B). Analysis of the cellular content within the lesion site indicated reduced inflammation levels and a higher number of neurons with elevated expression of markers associated with sprouting of axons during development and regeneration (FIGS. 5E-J). These results were translated into a significantly higher level of functional recovery, as judged by the sensorimotor functional analyses (FIGS. 5K-M).

Thus, according to a first aspect of the present invention, there is provided a composition comprising a plurality of fibrous particles fabricated from decellularized omentum, the fibrous particles being between 750 microns-3 mm in diameter, wherein the fibrous particles comprise a network of mature neurons.

The phrase “fibrous particles” refers to non-liquid particles fabricated from fibers of decellularized omentum which includes collagen and/or elastin fibers.

The fibrous particles described herein serve as a scaffold for the generation of the neuronal network.

As used herein, the term “scaffold” refers to a three dimensional structure comprising a biocompatible material that provides a surface suitable for adherence and proliferation of cells. A scaffold may further provide mechanical stability and support.

The fibers of the decellularized omentum which are comprised in the particle are typically between 50-200 nm in diameter, more typically between 50-150 nm in diameter, more typically 60-120 nm in diameter.

The particles of this aspect of the present invention are typically round, and more specifically substantially spherical, such as, spherical, oval, semi-spherical, hemispherical, an irregular sphere with flattened sections or concave or convex sections, semi-oval, an irregular oval with flattened sections or concave or convex sections.

The average diameter of the particles is typically being between 750 microns-3 mm, more typically between 1-2 mm.

According to a specific embodiment, the average diameter of the particles is greater than 500 microns.

As used herein, the term “particle size” refers to the particle size as determined, for example, by a laser scattering particle size distribution analyzer.

As used herein the phrase “decellularized omentum” refers to the extracellular matrix (ECM) which supports omentum tissue organization which has undergone a decellularization process (i.e., a removal of all cells from the tissue) and is thus devoid of cellular components.

The decellularized omentum comprises extracellular matrix (ECM) components.

The phrase “extracellular matrix (ECM)” as used herein, refers to a complex network of materials produced and secreted by the cells of the tissue into the surrounding extracellular space and/or medium and which typically together with the cells of the tissue impart the tissue its mechanical and structural properties. Generally, the ECM includes fibrous elements (particularly collagen, elastin, and/or reticulin), cell adhesion polypeptides (e.g., fibronectin, laminin and/or adhesive glycoproteins), and space-filling molecules [usually glycosaminoglycans (GAG), proteoglycans].

Omentum may be harvested from mammalian species, such as human, swine, bovine, goat and the like. Following tissue harvesting, the tissue can be either placed in 0.9% saline for immediate processing or stored for later use, preferably at a temperature of about −20° C. to about −80° C.

According to one embodiment of the present invention, the decellularization is carried out by:
(a) exposing the omentum to a hypotonic solution;
(b) dehydrating the omentum following step (a);
(c) extracting fat from the dehydrated omentum using polar and non-polar extraction agents following step (b);
(d) rehydrating the dehydrated omentum following step (c); and
(e) extracting cells from the rehydrated omentum following step (d).

A hypotonic solution is one in which the concentration of electrolyte is below that in cells. In this situation osmotic pressure leads to the migration of water into the cells, in an attempt to equalize the electrolyte concentration inside and outside the cell walls.

Preferably, the hypotonic buffer used by the method according to this aspect of the present invention is 10 mM Tris solution at a pH of about 8.0 and includes approximately 0.1% (w/v) EDTA (5 mM EDTA).

The hypotonic buffer may comprise additional agents such as serine protease inhibitors (e.g. phenylmethanesulfonylfluoride or phenylmethylsulfonyl fluoride, PMSF) and/or anionic detergents such as sodium dodecyl sulphate (SDS).

According to this aspect of the present invention, the tissue is subjected to the hypotonic buffer for a time period leading to the biological effect, i.e., cell swelling and rupture.

Following hypotonic shock, the tissue may optionally be subjected to cycles of freeze-thawing.

The freeze/thaw process preferably comprises freezing the tissue at, for example between −10 to −80° C., and typically at −80° C. for between 2-24 hours and subsequently defrosting the tissue for about 2, 3 or 4 hours until it reaches room temperature or above (for example at 37° C.). This process is carried out at least once and preferably twice or three times in the presence of a hypotonic buffer.

Dehydration involves treating the omentum with one or more dehydration solvents, such one or more treatments of the omentum with a dehydration solvent(s) and/or such solvent(s) in solution with water. The one or more treatments may be sequential steps in the method performed with solutions having different ratios of dehydration solvent(s) to water, such as having gradually reduced amounts of water in the solution for each successive treatment and the final treatment may involve the use of pure solvent, i.e., solvent not in solution with water.

Low molecular weight organic solvents may be used for the dehydration solvent. In an embodiment, the dehydration solvent is one or more alcohols, such as those selected from the group consisting of methanol, ethanol, isopropanol, propanol and combinations thereof.

According to a particular embodiment, the omentum is dehydrated by rinsing once with 70% ethanol (for example for 10-60 minutes) and two to three times in 100% ethanol for 10-60 minutes each.

After dehydration, the fat may be extracted from the omentum using at least one polar solvent and one non-polar solvent, which may occur in one or more extraction steps.

Examples of non-polar solvents are non-polar organic solvents such as hexane, xylene, benzene, toluene, ethyl acetate and combinations thereof. Polar solvents useful for the extraction solvent include acetone, dioxane, acetonithle and combinations thereof. In an embodiment, the extraction solvent is selected from acetone, hexane, xylene and combinations thereof. Nonpolar solvents, include for example hexane, xylene and combinations thereof.

Fat extraction may be conducted in fat extraction steps by contacting the dehydrated omentum with the extraction solvents for varying periods of time.

Preferably, the polar lipids of the tissue are extracted by washing in the polar extraction agent (e.g. 100% acetone) between 10 minutes to 60 minutes. This may be repeated a number of times (e.g. three times). Then, the nonpolar lipids may be extracted by incubating in a mixture of nonpolar:polar agents (e.g. 60/40 (v/v) hexane:acetone solution (with 3 changes) or 60/40 (v/v) hexane:isopropanol solution (with 3 changes)) for about 24 hours.

After the fat extraction, the defatted omentum is optionally re-hydrated. The defatted omentum maybe re-hydrated by contacting the defatted omentum with a re-hydration solvent, such as alcohol or a solution of alcohol in water, such as an alcohol solution having from about 60% to about 70% alcohol. Low molecular weight alcohols, such as methanol, ethanol, isopropanol, propanol and combinations thereof may be used.

The defatted omentum is then decellularized. Any decellularization process known to one skilled in the art may be applied to decellularize the defatted omentum.

Exemplary methods of decellularizing omentum may be found in US Patent No. 20150202348 and WO2014/037942, the contents of which are incorporated herein by reference.

In an embodiment, the defatted omentum may be decellularized by solubilization of the nuclear and cytoplasmic components. For example, the defatted omentum may be immersed in a decellularization buffer, such as one having non-ionic detergent and metal salt dissolved in acid for a period of time, typically at least about 30 minutes. Non-ionic detergents useful in the invention include polysorbates, such as TWEEN 80, ethoxylated alcohols, such as TRITON® X-100, and polyethanols, such as HP 40 and IGEPAL CA-630 and combinations thereof. Metal salts that may be used include magnesium chloride, phosphate, acetate and citrate, and combinations thereof and these metal salts are typically dissolved in Tris-HCL.

According to another embodiment, the defatted omentum may be decellularized by enzymatic proteolytic digestion which digests cellular components within the tissue yet preserves the ECM components (e.g., collagen and elastin) and thus results in a matrix which exhibits the mechanical and structural properties of the original tissue ECM. It will be appreciated that measures should be taken to preserve the ECM components while digesting the cellular components of the tissue. These measures are further described herein below and include, for example, adjusting the concentration of the active ingredient (e.g., trypsin) within the digestion solution as well as the incubation time.

Proteolytic digestion according to this aspect of the present invention can be affected using a variety of proteolytic enzymes. Non-limiting examples of suitable proteolytic enzymes include trypsin and pancreatin which are available from various sources such as from Sigma (St Louis, MO, USA). According to one preferred embodiment of this aspect of the present invention, proteolytic digestion is affected using trypsin.

Digestion with trypsin is preferably affected at a trypsin concentration ranging from 0.01-0.25% (w/v), more preferably, 0.02-0.2% (w/v), more preferably, 0.05-0.1 (w/v), even more preferably, a trypsin concentration of about 0.05% (w/v). For example, a trypsin solution containing 0.05% trypsin (w/v; Sigma), 0.02% EDTA (w/v) and antibiotics (Penicillin/Streptomycin, 1000 units/ml and 0.1 mg/mL respectively), pH=7.2] may be used to efficiently digest all cellular components of the tissue.

Preferably, while in the digestion solution, the tissue segments are slowly agitated (e.g., at about 150 rpm) to enable complete penetration of the digestion solution to all cells of the tissue.

It should be noted that the concentration of the digestion solution and the incubation time therein depend on the size of tissue segments utilized and those of skilled in the art are capable of adjusting the conditions according to the desired size and type of tissue.

Preferably, the tissue segments are digested for at least 1 hour and may be affected for up to 24 hours.

Following decellularization, the omentum may optionally be defatted again (e.g. using a combination of polar and non-polar solvents).

The method according to this aspect of the present invention optionally and preferably includes an additional step of removing nucleic acids (as well as residual nucleic acids) from the tissue to thereby obtain a nucleic acid-free tissue. As used herein the phrase “nucleic acid-free tissue” refers to a tissue being more than 99% free of any nucleic acid or fragments thereof as determined using conventional methods (e.g., spectrophotometry, electrophoresis). Such a step utilizes a DNase solution (and optionally also an RNase solution). Suitable nucleases include DNase and/or RNase [Sigma, Bet Haemek Israel, 20 μg/ml in Hank balance salt solution (HBSS)] or combinations of both—e.g. benzonase. High concentration of salts from 0.5M to 3M, such as sodium chloride, can be used also for nucleic acid elimination.

Next, the cellular components are typically removed from the tissue. Removal of the digested components from the tissue can be affected using various wash solutions, such as detergent solutions (e.g., ionic and non ionic detergents such as SDS Triton X-100, Tween-20, Tween-80) which can be obtained from e.g., Sigma (St Louis, MO, USA) or Biolab (Atarot, Israel, Merck Germany).

Preferably, the detergent solution used by the method according to this aspect of the present invention includes TRITON-X-100 (available from Merck). For efficient removal of all digested cellular components, TRITON-X-100 is provided at a concentration range of 0.05-2.5% (v/v), more preferably, at 0.05-2% (v/v), more preferably at 0.1-2% (v/v), even more preferably at a concentration of 1% (v/v).

Optionally, the detergent solution includes also ammonium hydroxide, which together with the TRITON-X-100, assists in breaking and dissolving cell nuclei, skeletal proteins, and membranes.

Preferably, ammonium hydroxide is provided at a concentration of 0.05-1.5% (v/v), more preferably, at a concentration of 0.05-1% (v/v), even more preferably, at a concentration of 0.1-1% (v/v) (e.g., 0.1%).

The concentrations of TRITON-X-100 and ammonium hydroxide in the detergent solution may vary, depending on the type and size of tissue being treated and those of skills in the art are capable of adjusting such concentration according to the tissue used.

Incubation of the tissue (or tissue segments) with the detergent solution can last from a few minutes to hours to even several days, depending on the type and size of tissue and the concentration of the detergent solution used and those of skills in the art are capable of adjusting such incubation periods. Preferably, incubation with the detergent solution is affected for at least 1 hour. According to one embodiment, 1-4 cycles of incubation with the detergent solution are performed until no foam is observed.

The above described detergent solution is preferably removed by subjecting the matrix to several washes in water or saline (e.g., at least 3 washes), until there is no evidence of detergent solution in the matrix.

Optionally, the decellularized ECM is then sterilized. Sterilization of the decellularized ECM may be affected using methods known in the art. In an embodiment, the decellularized omentum is contacted with a disinfection solution for a sufficiently effective period of time to disinfect the decellularized omentum, such as at least about 0.5 hour, typically about 1 hour to about 12 hours. The decellularized omentum may be fully submerged in the disinfection solution. The disinfection solution may comprise alcohol, or an alcohol in water solution, and may also include acid. The disinfection solution may include one or more of the following ethanol, methanol, isopropanol, propanol, hydrogen peroxide, peracetic acid and combinations thereof. In an embodiment, the disinfection solution has ethanol, such as 70% ethanol solution. Optionally, the decellularized omentum can be washed one or more times with ultrapure water.

Following washing and optional sterilization, the decellularized tissue may then be dehydrated for example by lyophilization.

Other methods contemplated by the present inventors for decellularizing tissue include those described in U.S. Pat. Nos. 4,776,853, 4,801,299 and U.S. Patent Publication No. 20090163990, the contents of each being incorporated herein by reference in their entirety.

The decellularized omentum of this aspect of the present invention typically comprises less than 20% of the cells as compared to the amount of cells in the omentum prior to decellularization, more preferably less than 15% of the cells as compared to the amount of cells in the omentum prior to decellularization, more preferably less than 10% of the cells as compared to the amount of cells in the omentum prior to decellularization, more preferably less than 5% of the cells as compared to the amount of cells in the omentum prior to decellularization, more preferably less than 2% of the cells as compared to the amount of cells in the omentum prior to decellularization.

In one embodiment, the decellularized omentum is devoid of cellular components.

The phrase “devoid of cellular components” as used herein refers to being more than 90%, 91%, 92%, 93%, 94%, 95%, 96%, 97%, 98%, 99%, (e.g., 100%) devoid of the cellular components present in the natural (e.g., native) omentum.

As used herein, the phrase “cellular components” refers to cell membrane components or intracellular components which make up the cell. Examples of cell components include cell structures (e.g., organelles) or molecules comprised in same. Examples of such include, but are not limited to, cell nuclei, nucleic acids, residual nucleic acids (e.g., fragmented nucleic acid sequences), cell membranes and/or residual cell membranes (e.g., fragmented membranes) which are present in cells of the tissue. It will be appreciated that due to the removal of all cellular components from the tissue, such a decellularized matrix cannot induce an immunological response when implanted in a subject.

The decellularized omentum of this aspect of the present invention is essentially devoid of lipids. The present inventors have found that the extent of extraction of lipids from the tissue correlates with the ability to induce cell attachment, maintain cell viability and promote proper assembly of cells into tissues.

The phrase “devoid of lipids” as used herein refers to a composition comprising less than 10%, 9%, 8%, 7%, 6%, 5%, 4%, 3%, 2%, 1% of the lipids present in the natural (e.g., native) omentum.

In order to generate particles fabricated from decellularized omentum, the decellularized omentum is solubilized.

Solubilization of the decellularized ECM may be affected as described in Freytes et al., Biomaterials 29 (2008) 1630-1637 and U.S. Patent Application No. 20120156250, the contents of which are incorporated herein by reference.

Typically, in order to carry out solubilization of the decellularized omentum it is first dehydrated e.g. lyophilized.

The lyophilized, decellularized omentum may be cut into small pieces, e.g. crumbled, or milled into a powder and then subjected to a second round of proteolytic digestion. The digestion is affected under conditions that allow the proteolytic enzyme to digest and solubilize the ECM. Thus, according to one embodiment, the digestion is carried out in the presence of an acid (e.g. hydrochloric acid) so as to obtain a pH of about 1-4.

Proteolytic digestion according to this aspect of the present invention can be affected using a variety of proteolytic enzymes. Non-limiting examples of suitable proteolytic enzymes include trypsin, pepsin, collagenase and pancreatin which are available from various sources such as from Sigma (St Louis, MO, USA) and combinations thereof. Matrix degrading enzymes such as matrix metalloproteinases are also contemplated.

It should be noted that the concentration of the digestion solution and the incubation time therein depend on the type of tissue being treated and the size of tissue segments utilized and those of skilled in the art are capable of adjusting the conditions according to the desired size and type of tissue.

Preferably, the tissue segments are incubated for at least about 20 hours, more preferably, at least about 24 hours. Preferably, the digestion solution is replaced at least once such that the overall incubation time in the digestion solution is at least 40-48 hours.

Once the decellularized omental ECM is solubilized, the pH of the solution is increased so as to irreversibly inactivate the proteolytic enzyme (e.g. to about pH 7). The decellularized, solubilized omentum may be stored at this stage at temperatures lower than 20° C.—for example 4° C. so that the decellularized ECM remains in solution.

The solubilized, decellularized omentum is capable of forming a gel at a temperature above about 30° C., above about 31° C., above about 32° C., above about 33° C., above about 34° C., above about 35° C., above about 36° C., above about 37° C.

In order to generate the particles of this aspect of the present invention, solubilized, decellularized omentum is typically mixed with stem cells that are capable of differentiating into neurons and form a neuronal network.

Below is a description of various stem cells which can be used to generate the particles of this aspect of the present invention.

The stem cells may be genetically modified or non-genetically modified. For example, the cells may be genetically modified to express an exogenous polypeptide or polynucleotide (e.g. an RNA silencing agent such as siRNA).

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells). Totipotent cells, such as embryonic cells within the first couple of cell divisions after fertilization are the only cells that can differentiate into embryonic and extra-embryonic cells and are able to develop into a viable human being. Preferably, the phrase “pluripotent stem cells” refers to cells which can differentiate into all three embryonic germ layers, i.e., ectoderm, endoderm and mesoderm or remaining in an undifferentiated state. The pluripotent stem cells include embryonic stem cells (ESCs) and induced pluripotent stem cells (iPSCs). The multipotent stem cells include adult stem cells and hematopoietic stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

Induced pluripotent stem cells (iPSCs; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as omentum) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in omental cells.

The phrase “adult stem cells” (also called “tissue stem cells” or a stem cell from a somatic tissue) refers to any stem cell derived from a somatic tissue [of either a postnatal or prenatal animal (especially the human)]. The adult stem cell is generally thought to be a multipotent stem cell, capable of differentiation into multiple cell types. Adult stem cells can be derived from any adult, neonatal or fetal tissue such as adipose tissue, skin, kidney, liver, prostate, pancreas, intestine, bone marrow and placenta.

Hematopoietic stem cells, which may also referred to as adult tissue stem cells, include stem cells obtained from blood or bone marrow tissue of an individual at any age or from cord blood of a newborn individual. Preferred stem cells according to this aspect of some embodiments of the invention are embryonic stem cells, preferably of a human or primate (e.g., monkey) origin.

Placental and cord blood stem cells may also be referred to as “young stem cells”.

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used according to some embodiments of the invention. Human ES cells can be purchased from the NIH human embryonic stem cells registry [Hypertext Transfer Protocol://grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Encavour-2, WIBR1, WIBR2, WIBR3, WIBR4, WIBR5, WIBR6, HUES 45, Shef 3, Shef 6, BJNhem19, BJNhem20, SA001, SA001.

In addition, ES cells can be obtained from other species as well, including mouse (Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, MO, USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Another method for preparing ES cells is described in Chung et al., Cell Stem Cell, Volume 2, Issue 2, 113-117, 7 Feb. 2008. This method comprises removing a single cell from an embryo during an in vitro fertilization process. The embryo is not destroyed in this process.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Embryonic stem cells (e.g., human ESCs) originating from an unfertilized ova stimulated by parthenogenesis (parthenotes) are known in the art (e.g., Zhenyu Lu et al., 2010. J. Assist Reprod. Genet. 27:285-291; “Derivation and long-term culture of human parthenogenetic embryonic stem cells using human foreskin feeders”, which is fully incorporated herein by reference). Parthenogenesis refers to the initiation of cell division by activation of ova in the absence of sperm cells, for example using electrical or chemical stimulation. The activated ovum (parthenote) is capable of developing into a primitive embryonic structure (called a blastocyst) but cannot develop to term as the cells are pluripotent, meaning that they cannot develop the necessary extra-embryonic tissues (such as amniotic fluid) needed for a viable human foetus.

According to a particular embodiment, the iPSCs cells are reprogrammed (i.e. dedifferentiated) from omental stromal cells.

Adult tissue stem cells can be isolated using various methods known in the art such as those disclosed by Alison, M. R. [J Pathol. 2003 200(5): 547-50], Cai, J. et al., [Blood Cells Mol Dis. 2003 31(1): 18-27], Collins, A. T. et al., [J Cell Sci. 2001; 114(Pt 21): 3865-72], Potten, C. S. and Morris, R. J. [Epithelial stem cells in vivo. 1988. J. Cell Sci. Suppl. 10, 45-62], Dominici, M et al., [J. Biol. Regul. Homeost. Agents. 2001, 15: 28-37], Caplan and Haynesworth [U.S. Pat. No. 5,486,359] Jones E. A. et al., [Arthritis Rheum. 2002, 46(12): 3349-60]. Fetal stem cells can be isolated using various methods known in the art such as those disclosed by Eventov-Friedman S, et al., PLOS Med. 2006, 3: e215; Eventov-Friedman S, et al., Proc Natl Acad Sci USA. 2005, 102: 2928-33; Dekel B, et al., 2003, Nat Med. 9: 53-60; and Dekel B, et al., 2002, J. Am. Soc. Nephrol. 13: 977-90. Hematopoietic stem cells can be isolated using various methods known in the arts such as those disclosed by “Handbook of Stem Cells” edit by Robert Lanze, Elsevier Academic Press, 2004, Chapter 54, pp 609-614, isolation and characterization of hematopoictic stem cells, by Gerald J Spangrude and William B Stayton.

Generally, isolation of adult tissue stem cells is based on the discrete location (or niche) of each cell type included in the adult tissue, i.e., the stem cells, the transit amplifying cells and the terminally differentiated cells [Potten, C. S. and Morris, R. J. (1988). Epithelial stem cells in vivo. J. Cell Sci. Suppl. 10, 45-62]. Thus, an adult tissue such as, for example, prostate tissue is digested with Collagenase and subjected to repeated unit gravity centrifugation to separate the epithelial structures of the prostate (e.g., organoids, acini and ducts) from the stromal cells. Organoids are then disaggregated into single cell suspensions by incubation with Trypsin/EDTA (Life Technologies, Paisley, UK) and the basal, CD44-positive, stem cells are isolated from the luminal, CD57-positive, terminally differentiated secretory cells, using anti-human CD44 antibody (clone G44-26; Pharmingen, Becton Dickinson, Oxford, UK) labeling and incubation with MACS (Miltenyi Biotec Ltd, Surrey, UK) goat anti-mouse IgG microbeads. The cell suspension is then applied to a MACS column and the basal cells are eluted and re-suspended in WAJC 404 complete medium [Robinson, E. J. et al. (1998). Basal cells are progenitors of luminal cells in primary cultures of differentiating human prostatic epithelium Prostate 37, 149-160].

Since basal stem cells can adhere to basement membrane proteins more rapidly than other basal cells [Jones, P. H. et al. (1995). Stem cell patterning and fate in human epidermis. Cell 60, 83-93; Shinohara, T., et al. (1999). β1- and α6-integrin are surface markers on mouse spermatogonial stem cells. Proc. Natl. Acad. Sci. USA 96, 5504-5509] the CD44 positive basal cells are plated onto tissue culture dishes coated with either type I collagen (52 μg/ml), type IV collagen (88 μg/ml) or laminin 1 (100 μg/ml; Biocoat®, Becton Dickinson) previously blocked with 0.3% bovine serum albumin (fraction V, Sigma-Aldrich, Poole, UK) in Dulbecco's phosphate buffered saline (PBS; Oxoid Ltd, Basingstoke, UK). Following 5 minutes, the tissue culture dishes are washed with PBS and adherent cells, containing the prostate tissue basal stem cells are harvested with trypsin-EDTA.

### BM-Derived Stem Cell, Mesenchymal Stem Cells

In one embodiment, the stem cells utilized by some embodiments of the invention are BM-derived stem cells including hematopoietic, stromal or mesenchymal stem cells (Dominici, M et al., 2001. Bone marrow mesenchymal cells: biological properties and clinical applications. J. Biol. Regul. Homeost. Agents. 15: 28-37). BM-derived stem cells may be obtained from iliac crest, femora, tibiac, spine, rib or other medullar spaces.

Of the above described BM-derived stem cells, mesenchymal stem cells are the formative pluripotent blast cells. Mesenchymal stem cells give rise to one or more mesenchymal tissues (e.g., adipose, osscous, cartilaginous, clastic and fibrous connective tissues, myoblasts) as well as to tissues other than those originating in the embryonic mesoderm (e.g., neural cells) depending upon various influences from bioactive factors such as cytokines. Although such cells can be isolated from embryonic yolk sac, placenta, umbilical cord, fetal and adolescent skin, blood and other tissues, their abundance in the BM far exceeds their abundance in other tissues and as such isolation from BM is presently preferred.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

Preferably, mesenchymal stem cell cultures are generated by diluting BM aspirates (usually 20 ml) with equal volumes of Hank's balanced salt solution (HBSS; GIBCO Laboratories, Grand Island, NY, USA) and layering the diluted cells over about 10 ml of a Ficoll column (Ficoll-Paque; Pharmacia, Piscataway, NJ, USA). Following 30 minutes of centrifugation at 2,500×g, the mononuclear cell layer is removed from the interface and suspended in HBSS. Cells are then centrifuged at 1,500×g for 15 minutes and resuspended in a complete medium (MEM, a medium without deoxyribonucleotides or ribonucleotides; GIBCO); 20% fetal calf serum (FCS) derived from a lot selected for rapid growth of MSCs (Atlanta Biologicals, Norcross, GA); 100 units/ml penicillin (GIBCO), 100 μg/ml streptomycin (GIBCO); and 2 mM L-glutamine (GIBCO). Resuspended cells are plated in about 25 ml of medium in a 10 cm culture dish (Corning Glass Works, Corning, NY) and incubated at 37° C. with 5% humidified CO₂. Following 24 hours in culture, nonadherent cells are discarded, and the adherent cells are thoroughly washed twice with phosphate buffered saline (PBS). The medium is replaced with a fresh complete medium every 3 or 4 days for about 14 days. Adherent cells are then harvested with 0.25% trypsin and 1 mM EDTA (Trypsin/EDTA, GIBCO) for 5 min at 37° C., replated in a 6-cm plate and cultured for another 14 days. Cells are then trypsinized and counted using a cell counting device such as for example, a hemocytometer (Hausser Scientific, Horsham, PA). Cultured cells are recovered by centrifugation and resuspended with 5% DMSO and 30% FCS at a concentration of 1 to 2× 10⁶ cells per ml. Aliquots of about 1 ml each are slowly frozen and stored in liquid nitrogen.

To expand the mesenchymal stem cell fraction, frozen cells are thawed at 37° C., diluted with a complete medium and recovered by centrifugation to remove the DMSO. Cells are resuspended in a complete medium and plated at a concentration of about 5,000 cells/cm². Following 24 hours in culture, nonadherent cells are removed and the adherent cells are harvested using Trypsin/EDTA, dissociated by passage through a narrowed Pasteur pipette, and preferably replated at a density of about 1.5 to about 3.0 cells/cm². Under these conditions, MSC cultures can grow for about 50 population doublings and be expanded for about 2000 fold [Colter D C., et al. Rapid expansion of recycling stem cells in cultures of plastic-adherent cells from human bone marrow. Proc Natl Acad Sci USA. 97: 3213-3218, 2000].

MSC cultures utilized by some embodiments of the invention preferably include three groups of cells which are defined by their morphological features: small and agranular cells (referred to as RS-1, hereinbelow), small and granular cells (referred to as RS-2, hereinbelow) and large and moderately granular cells (referred to as mature MSCs, hereinbelow). The presence and concentration of such cells in culture can be assayed by identifying a presence or absence of various cell surface markers, by using, for example, immunofluorescence, in situ hybridization, and activity assays.

When MSCs are cultured under the culturing conditions of some embodiments of the invention they exhibit negative staining for the hematopoietic stem cell markers CD34, CD11B, CD43 and CD45. A small fraction of cells (less than 10%) are dimly positive for CD31 and/or CD38 markers. In addition, mature MSCs are dimly positive for the hematopoietic stem cell marker, CD117 (c-Kit), moderately positive for the osteogenic MSCs marker, Stro-1 [Simmons, P. J. & Torok-Storb, B. (1991). Blood 78, 5562] and positive for the thymocytes and peripheral T lymphocytes marker, CD90 (Thy-1). On the other hand, the RS-1 cells are negative for the CD117 and Stro1 markers and are dimly positive for the CD90 marker, and the RS-2 cells are negative for all of these markers.

As mentioned, in order to generate the particles of the present invention, solubilized, decellularized omentum is mixed with the stem cells (e.g. dissociated colonies of iPSCs cells). Droplets of between 1-10 μL, for example between 2-5 μL of the solution may be generated using a drop-making device (e.g. pipette) onto a solid surface, such as silicon glass or plastic. Other surface types are also envisaged including oil based surfaces and water based surfaces. The droplets are generated at a temperature which maintains the decellularized omentum as a liquid. Once formed, the droplets are then subjected to a temperature of above 30° C. (e.g. 37° C.) for at least half an hour to ensure that the droplets have solidified and form solid, gel-like particles. Upon gelation the particles are then cultured in a medium, such that the cells seeded therein remain viable.

In one embodiment, the droplets (or particles) are not subjected to chemical crosslinkers.

In another embodiment, the droplets (or particles) are subjected to additional crosslinkers.

Exemplary chemical crosslinkers such as Carbodiimides (EDC and DCC), N-Hydroxysuccinimide Esters (NHS Esters), Imidoesters, Maleimides, Haloacetyls, Pyridyl Disulfides, Hydrazides, Alkoxyamines, Aryl Azides, Diazirines, Staudinger Reagent Pairs are contemplated.

Alternatively, or additionally, enzymes such as transglutaminase, sortase, laccase/peroxidase, lysyl oxidase/amine oxidase may be used for crosslinking. Other enzymes are disclosed in Heck et al., Appl Microbiol Biotechnol. 2013 January; 97(2): 461-475, the contents of which are incorporated by reference.

Additional chemical crosslinkers that may be used for the present invention include Carbodiimides (EDC and DCC), N-Hydroxysuccinimide Esters (NHS Esters), Imidoesters, Malcimides, Haloacetyls, Pyridyl Disulfides, Hydrazides, Alkoxyamines, Aryl Azides, Diazirines, Staudinger Reagent.

Other method of forming particles of decellularized omentum are known in the art and include those disclosed in US Patent Application No. 20180361023, the contents of which are incorporated herein by reference.

It will be appreciated that since the cells are mixed with the decellularized omentum when it is in a liquid form (i.e. prior to particle formation) and not seeded upon the pre-formed particles, the cells are typically distributed homogeneously throughout the particles.

Prior to the differentiation step, the stem cells comprised in the particle may be allowed to proliferate to fill the volume—e.g. for at least 1 day, 3 days, 7 days or more. The particles are cultured in a medium which prevents the differentiation of the cells i.e. serves to maintain their pluripotency. Typically, each particle comprises about 15,000-150,000 stem cells at the start of the differentiation step. In one embodiment, the differentiation step is started when the cells reach about 90% confluence.

In one embodiment, the particles are cultured in a medium comprising neuronal differentiating agents under conditions that promote diffusion of the neuronal differentiating agents into the particle.

Methods of differentiating pluripotent stem cells into neuronal cells are known in the art and include those disclosed by Edri et al., Advanced materials 31, 1803895 (2019); Shimojo, et al. Mol Brain 8, 79 (2015): Yi et al., Stem Cells International. 2018, Article ID 3628578; Faravelli et al. Stem Cell Research & Therapy, 2014, 5:87; Wada et al. PLOS One, 2009, Volume 4, Issue 8, e6722; Qu et al., Nature Communications, 5:3449|DOI: 10.1038/ncomms4449; Karumayaram et al., Stem Cells. 2009 April; 27(4): 806-811. doi: 10.1002/stem.31, the contents of each are incorporated herein by reference.

Methods of differentiating mesenchymal stem cells into cells of the neuronal lineage are provided for example in WO2006/134602, WO2009/144718, WO2007/066338 and WO2004/046348, the teachings of which are incorporated herein by reference.

Exemplary neuronal differentiation agents which can be used in the differentiation process include, but are not limited to retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-4, jagged 1 antagonist, jagged 2 antagonist; numb agonist or numb-like agonist.

According to one embodiment, the culturing is carried out under conditions that promote differentiation of the cells in the particle to mature neurons (e.g. mature motor neurons) which form a neuronal network in the particle.

The neurons may be excitatory neurons or inhibitory neurons.

In one embodiment, the neurons comprise motor neurons.

The neurons of this aspect of the present invention express markers indicative of mature neurons (e.g. express dendritic markers such as MAP2, markers for synapses (SYP) and markers for neuronal intermediate filaments (NFM).

In another embodiment, the neurons express markers of mature motor neurons including, but not limited to choline acetyltransferase (ChAT), HB9 (also known as MNX1) and ISL-1.

Preferably at least 20%, 30%, 40%, 50%, 60%, 70%, 80%, 90% of the cells of the neuronal network in the particle express a marker for mature motor neurons, as determined by immunofluorescence or flow cytometry analysis.

For example, in one embodiment, more than 50%, 60%, 70%, 80%, 90% of the motor neurons in the particles express Neuron-specific class III beta-tubulin (TUJ1), as measured by flow cytometry.

According to another embodiment, more than 50%, 60%, 70%, 80%, 90% of the motor neurons in the particle express Motor neuron and pancreas homeobox 1 (MNX1), as measured by flow cytometry.

In still another embodiment, the neurons in the network are capable of displaying synchronous neural firing in vitro.

The term “neuronal network” refers to a collection of interconnected neurons comprising dendrites and having synapses therebetween. The neurons of the neuronal network in a single particle are capable of interacting with neurons of a neuronal network of a second particle (under appropriate conditions). In one embodiment, the neuronal network also comprises neurofilaments.

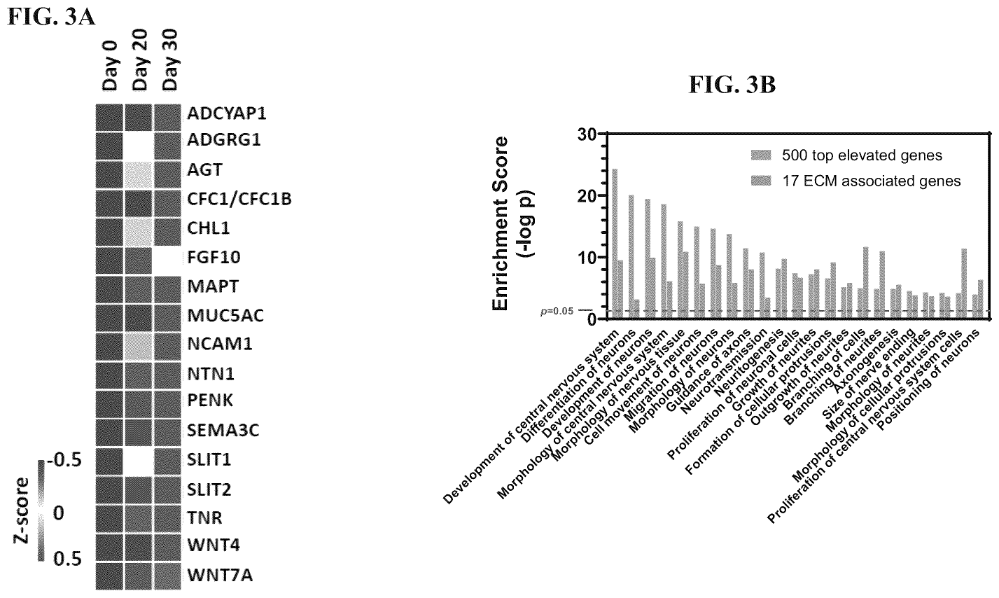

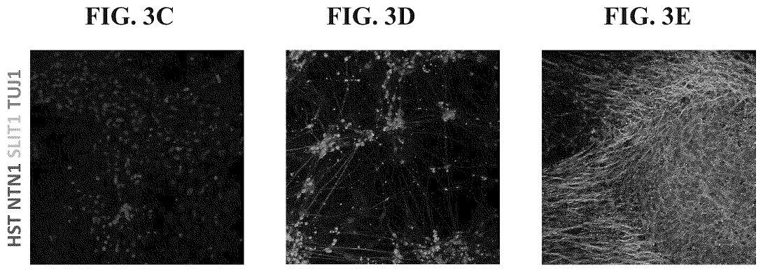

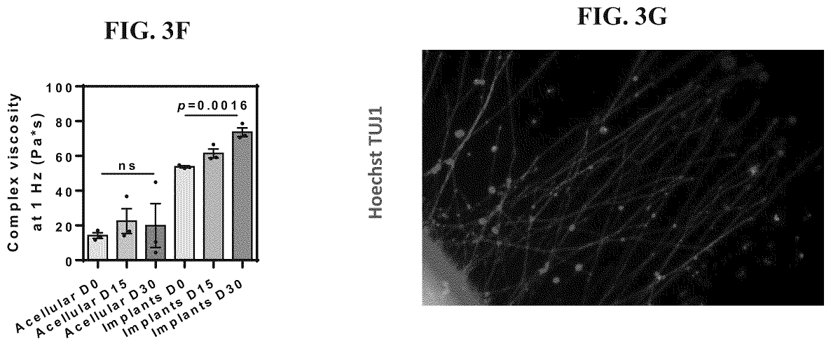

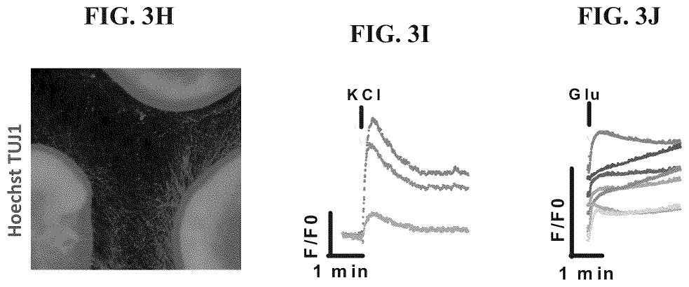

The neurons of the network in a particular particle are capable of connecting with neurons of the network of another particle, under appropriate conditions. In one embodiment, the connections between the neurons of the two particles occurs following transplantation into the site of injury. In another embodiment, the connections between the neurons of the two particles can occur ex vivo (see for example FIGS. 3G and 3H).

In one embodiment, the differentiation process comprises:
(a) culture iPSCs in the presence of an ALK5 inhibitor, an ALK2/ALK3 inhibitor and a GSK3 inhibitor
(b) subsequent culture in the presence of retinoic acid and a hedgehog pathway agonist (purmorphamine)
(c) subsequent culture in the presence of sonic hedgehog and retinoic acid;
(d) subsequent culture in the presence of a neurotrophic factor (e.g. BDNF), ascorbic acid, hedgehog pathway agonist (purmorphamine) and retinoic acid; and
(e) subsequent culture in the presence of a γ-secretase inhibitor (e.g. DAPT).

The particles may also comprise additional cells such as astrocytes.

Therapeutic compounds or agents that modify cellular activity can also be incorporated (e.g. attached to, coated on, embedded or impregnated) into the particles. Campbell et al (US Patent Application No. 20030125410) which is incorporated by reference as if fully set forth by reference herein, discloses methods for fabrication of 3D scaffolds for stem cell growth, the scaffolds having preformed gradients of therapeutic compounds. The scaffold materials, according to Campbell et al, fall within the category of “bio-inks”. Such “bio-inks” are suitable for use with the compositions and methods of the present invention.

Exemplary agents that may be incorporated into the particles of the present invention include, but are not limited to those that promote cell adhesion (e.g. fibronectin, integrins), cell colonization, cell proliferation, cell differentiation, anti-inflammatoires, cell extravasation and/or cell migration. Thus, for example, the agent may be an amino acid, a small molecule chemical, a peptide, a polypeptide, a protein, a DNA, an RNA, a lipid and/or a protcoglycan.

Proteins that may be incorporated into the particles of the present invention include, but are not limited to extracellular matrix proteins, cell adhesion proteins, growth factors, cytokines, hormones, proteases and protease substrates. Thus, exemplary proteins include vascular endothelial-derived growth factor (VEGF), activin-A, retinoic acid, epidermal growth factor, bone morphogenetic protein, TGFβ, hepatocyte growth factor, platelet-derived growth factor, TGFα, IGF-I and II, hematopoetic growth factors, heparin binding growth factor, peptide growth factors, erythropoietin, interleukins, tumor necrosis factors, interferons, colony stimulating factors, basic and acidic fibroblast growth factors, nerve growth factor (NGF) or muscle morphogenic factor (MMP). The particular growth factor employed should be appropriate to the desired cell activity. The regulatory effects of a large family of growth factors are well known to those skilled in the art.

In one embodiment, only neuronal cells are comprised in the particles.

In still another embodiment, the particles are essentially devoid of pluripotent stem cells.

Preferably, at least 50%, 60%, 70%, 80%, 90% of the pluripotent stem cells have been differentiated towards a neuronal lineage and more preferably towards the mature neuronal cell type.

Thus, for example, no more than 1%, 3%, 5%, 10%, 15% or 20% of the cells express markers of pluripotency (e.g. TRA-1-60. SSEA4, OCT4), as measured by flow cytometry or immunohistochemistry.

The particles of the present invention may be used per se for the treatment of a spinal cord injury or as part of a pharmaceutical composition, where they are mixed with suitable carriers or excipients.

As used herein a “pharmaceutical composition” refers to a preparation of one or more of the particles described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Examples, without limitations, of carriers are propylene glycol; saline; emulsions; buffers; culture medium such as DMEM or RPMI; hypothermic storage medium containing components that scavenge free radicals, provide pH buffering, oncotic/osmotic support, energy substrates and ionic concentrations that balance the intracellular state at low temperatures; and mixtures of organic solvents with water. Typically, the pharmaceutical carrier preserves the number of particles (e.g. is not reduced by more than 90%) and maintains the viability of the cells in the particles in the composition for at least 24 hours, at least 48 hours or even at least 96 hours.

According to a specific embodiment, the physiologically acceptable carrier is saline.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Pharmaceutical compositions suitable for use in context of some embodiments of the invention include compositions wherein the active ingredients are contained in an amount effective to achieve the intended purpose. More specifically, a therapeutically effective amount means an amount of active ingredients (particles described herein) effective to prevent, alleviate or ameliorate symptoms of a disorder or injury (e.g., spinal cord injury) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is affected or diminution of the disease state is achieved.

The amount of a composition to be administered will, of course, be dependent on the subject being treated, the severity of the affliction, the manner of administration, the judgment of the prescribing physician, etc.

The particles of the present invention, in at least some embodiments, may be prepackaged in unit dosage forms in a syringe ready for use. The syringe may be labeled with the name of the particles and their source. The labeling may also comprise information related to the function of the particles. The syringe may be packaged in a packaging which is also labeled with information regarding the particles.

The particles described herein are useful for treating spinal cord injury.

Thus, according to another aspect of the present invention, there is provided a method of treating a chronic spinal cord injury of a subject comprising transplanting the composition described herein into the subject at the site of injury, at least three months following the spinal cord injury, thereby treating the spinal cord injury.

As used herein, the phrase “spinal cord injury” refers to an injury to the spinal cord that is caused by trauma instead of disease. Depending on where the spinal cord and nerve roots are damaged, the symptoms can vary widely, for example from pain to paralysis to incontinence. Spinal cord injuries are described at various levels of “incomplete”, which can vary from having no effect on the patient to a “complete” injury which means a total loss of function. Spinal cord injuries have many causes, but are typically associated with major trauma from motor vehicle accidents, falls, sports injuries, and violence. The abbreviation “SCI” means spinal cord injury.

The spinal cord injury may be susceptible to secondary tissue injury, including but not limited to: glial scarring, myelin inhibition, demyelination, cell death, lack of neurotrophic support, ischemia, free-radical formation, and excitotoxicity. This secondary tissue injury typically occurs at least 3 months, 4 months, five months, six months or later after the initial injury. This phase can also be referred to as chronic spinal cord injury.

According to a particular embodiment, the particles are transplanted to the site of injury following removal of the scar tissue. The particles are typically transplanted using a device suitable for administering the particles. In one embodiment, the device is a syringe (e.g. one having an inner diameter between 1-5 mm). Following injection of the particles to the site of injury, typically the carrier is suctioned back into the syringe.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

General references are provided throughout this document. The procedures therein are believed to be well known in the art and are provided for the convenience of the reader. All the information contained therein is incorporated herein by reference.

### Example 1

### Materials and Methods

Omentum hydrogel formation: Omentum decellularization (M. Shevach et al., Biomedical materials 10, 034106 (2015)): Pig omenta (Kibutz Lahav, Israel) were washed with phosphate buffered saline (PBS) and major blood vessels were removed. The samples were then moved to a hypotonic buffer (10 mM Tris, 5 mM ethylenediaminetetraacetic acid (EDTA) and 1 μM phenylmethanesulfonyl-fluoride (PMSF), pH 8.0) for 1 hour. Next, tissues were frozen and thawed 3 times using the same buffer. The tissues were washed gradually with 70% ethanol and 100% ethanol for 30 min each. Lipids were extracted by three 30 min washes of 100% acetone, followed by 24 hour incubation in a 60/40 (v/v) hexane: acetone solution (3 changes). The defatted tissue was washed in 100% ethanol for 30 min and incubated overnight at 4° C. in 70% ethanol. Then, the tissue was washed four times with PBS (pH 7.4) and incubated in 0.25% Trypsin-EDTA (Biological Industries) overnight. The tissue was washed thoroughly with PBS and incubated with 1.5 M NaCl for 24 h (3 changes), followed by washing in 50 mM Tris (pH 8.0), 1% triton-X100 (Sigma) solution for 1 hour. The decellularized tissue was washed in PBS followed by double distilled water and then frozen (−20° C.) and lyophilized.

Solubilization of omentum hydrogel: After lyophilization, decellularized omentum was ground into powder (Wiley Mini-Mill, Thomas Scientific, Swedesboro, NJ)). Dry, milled omentum was enzymatically digested for 96 h at room temperature in a 1 mg ml-1 solution of pepsin (Sigma, 4000 U mg⁻¹) in 0.1 M HCl, with stirring. Subsequently, the pH was adjusted to 7.4 using cither DMEM/F12 X10 or PBS X10 (Biological industries). The final concentration of decellularized omentum in the titrated solution was 1.5% (w/v). At least 10 pigs omenta were used.

Culturing undifferentiated iPSCs: iPSCs were generated from omental stromal cells. The undifferentiated cells were cultivated on culture plates, pre-coated with MATRIGEL™ (BD, New Jersey), diluted to 250 μg/mL in DMEM/F12 (Biological Industries, Beit HaEmek, Israel), or cultured within the omentum hydrogel. All cells were cultured at 37° C. with 5% CO₂. Undifferentiated iPSCs were maintained in NutriStem® (Biological Industries) medium containing 0.1% Penicillin/Streptomycin (Biological Industries). Medium was replaced daily and cells were passaged weckly with 1 U/mL dispase (Stemcell Technologies, Vancouver, Canada) followed by mechanical trituration. iPSCs were seeded in small colonies in the presence of Y-27632 (10 μM; Tocris, UK).

Spinal cord motor neurons implants generation and differentiation: Dissociated iPSCs colonies were mixed with 1.5% omentum-based hydrogel at equal volumes. Droplets of 3 μL were generated using a pipette. The implants were crosslinked at 37° C. on a damp towel for 30 min after which culture medium was added. Undifferentiated cells were cultured in Nutristem that was replaced daily, until 90% confluence was achieved. Cells were differentiated as previously described (R. Edri et al., Advanced materials 31, 1803895 (2019). Briefly, after achieving ˜90% confluence, medium was changed to Knockout/DMEM, supplemented with 15% Knockout Scrum, 0.1% penicillin/streptomycin, 0.5%1-glutamine, 1% non-essential amino acids (Invitrogen), 10 μM β-mercaptocthanol, 10 mM SB-431542 (Tocris), 1 μM LDN-193189 (Tocris), and 3 μM CHIR-99021 and was gradually changed every 3 days to DMEM/F12 supplemented with N2 (Day 3 was ¾ Knockout/DMED and ¼ DMEM/F12 with N₂ supplemented for the F12 portion only, day 6 was changed as ½ ½). On days 4 and 6, the motor neuron medium was supplemented with 1 μM retinoic acid and 1 μM purmorphamine (Tocris). On day 8, DMEM F/12 supplemented with N2, 30 ng/ml sonic hedgehog (R&D) and 1 μM retinoic acid was added to the cells (⅓ of the final volume, without changing medium). After day 10, the medium was changed to DMEM/F12 supplemented with N2, 0.1% P/S, 5 μg/mL BDNF (R&D), 200 μM ascorbic acid (Sigma), 1 μM purmorphamine (Tocris) and 1 μM retinoic acid. From day 15, 5 μM DAPT (Tocris) was also added, and purmorphamine concentration was decreased to 500 nM. Medium was changed every 3 days until day 30.

Immunostaining and confocal imaging: Cellular implants were fixed in 4% formaldehyde, permeabilized with 0.05% (v/v) triton X-100 and blocked with PBS, 1% bovine serum albumin (BSA), 10% fetal bovine serum (FBS) and stained with the indicated primary antibodies followed by secondary antibodies (as presented in the Antibody list, Supporting Information). Cells and implants were imaged using an upright confocal microscope (Nikon ECLIPSE NI-E) and inverted fluorescence microscope (Nikon ECLIPSE TI-E). Images were processed and analyzed using NIS elements software (Nikon Instruments).

Neurite outgrowth assay: For neurite outgrowth assay, implants at day 30 of differentiation were placed on 250 μg/mL MATRIGEL™-coated plates. The constructs were cultured for 3 days before fixation in 4% formaldehyde and imaging using inverted fluorescence microscope (Nikon ECLIPSE TI-E).

RNA seq and bioinformatics analysis: RNA samples of implants on days 0, 20 and 30 were extracted using miRNeasy kit (Qiagen, Hilden, Germany) and were treated with DNase (Qiagen). Pooled samples from at least 2 different experimental replicates were quantified. Library quality control was performed using FASTQC (version 0.11.5) followed by quality and adapter trimming using Cutadapt, Version 1.1 (M. Martin, EMBnet. journal 17, 10-12 (2011)). All reads were aligned to the Homo sapiens reference genome using the TopHat aligner (Trapnell) with a maximal mismatch parameter of 3 and minimum and maximum intron sizes of 70 and 500000, respectively. The raw expression levels were calculated using HTseq-count, version 0.6.1 (S. Anders, P. T. Pyl, W. Huber, bioinformatics 31, 166-169 (2015)). Reads per kilobase million (RPKM) values were then calculated based on the raw expression levels using annotations from Ensembel gene reference file (version GRCh38.87).

Functional enrichments were analyzed using the Ingenuity® Pathway Analysis (IPA®, Qiagen Bioinformatics, Redwood City, CA) software for the 500 protein-coding genes with the highest increasing expression based on the RPKM fold-change (day 30 of differentiation normalized to undifferentiated iPSCs) in engineered spinal cord tissue. For ECM analyses, 17 ECM-associated genes were chosen from the above 500, and functional enrichment was performed. Functions that were chosen for ECM analyses, were associated with at least two out of the 17 ECM genes. Z-scores for each sample separately were calculated according to RPKM values. Complete RNA data is available under accession number GSE97341.

Calcium imaging: For calcium imaging, the implants were incubated with 10 μM fluo-4 AM (Invitrogen) and 0.1% Pluronic F-127 (Sigma-Aldrich) for 45 min at 37° C. Implants were then washed in Hank's buffer salts solution (HBSS) and imaged using an inverted fluorescent microscope (Nikon Eclipse TI). Videos were acquired with an ORCA-Flash 4.0 digital complementary metal-oxide semiconductor (CMOS) camera (Hamamatsu) at 2 frames/s. HBSS was used as the external solution. Baseline was recorded for 30 sec, after which depolarization was induced by KCl (50 mM; final concentration 25 mM) or glutamate (200 μM; final concentration 100 μM) solutions. Injection of solutions was performed in situ, allowing a non-interrupted recording of the cellular Ca²⁺ responses. The data was analyzed using ImageJ (NIH) and normalized by dividing each data set by the first value (F/F₀).

Spinal cord hemisection: Hemisection was performed as previously described (Y. Goldshmit et al., J Neurosci 24, 10064-10073 (2004). Briefly, mice (20-30 g) were anaesthetized with intraperitoneally-injected ketamine (100 mg/kg) and xylazine (16 mg/kg) in PBS. The spinal cord was exposed at the low thoracic to high lumbar area. After laminectomy, a complete left hemisection was made at T10 and the overlying muscle and skin were sutured. In acute phase injury, mice were immediately treated. The controls groups were: Untreated group which was treated with 10 μL saline; Cells group, treated with dissociated iPSCs-derived spinal cord neurons (day 30 of differentiation) suspended in 10 μL saline; and Hydrogel group that was treated with 0.75% pre-crosslinked omentum-based hydrogel. The tested treatment was applied to the Implants group which was treated with differentiated iPSCs-derived spinal cord neuron implants (day 30 of differentiation).

In chronic phase injury model, SCI was induced similarly to the acute phase. Six weeks after the initial SCI, animals were re-anesthetized, and the lesion area was re-opened. Scar was resected carefully, and treatments were applied in the cavity formed by the ablated scar. Mice were randomly assigned to the four groups and allowed to survive for 1 week to 3 months post injury.

Tissue preparation and immunofluorescence labeling. One-, eight- or twelve-weeks post-treatment, the animals were anesthetized and transcardially perfused with 20 mL PBS (pH 7.4) followed by 20 mL 4% paraformaldehyde. Spinal cord tissue was dissected, post-fixed in 4% paraformaldehyde for 24 h at 4° C. and dehydrated with 20% v/v sucrose overnight at 4° C. The dissected tissues were embedded in OCT and cut longitudinally into 60-μm thick cryosections using a freezing microtome (Leica CM1950, Germany).

For immunostaining, sections were permeabilized with 0.3% Triton X-100 in PBS solution, blocked with 10% FBS and 1% BSA in PBS for 1 h, and then incubated with primary antibodies (Antibody list, Supporting Information) overnight at 4° C. After rinsing with PBS three times, the sections were incubated with Alexa Fluor 488/594/647 conjugated secondary antibodies for 1 h. Then, the sections were washed three times with PBS, counterstained with Hoechst 33528 (5 μg/mL) for 10 min at room temperature, then washed with PBS, left to dry and covered with glass slips in an anti-fade fluorescent mounting medium. The sections were stored at 4° C. until characterized using a Nikon confocal microscope. The integrated density of each epicenter was automatically calculated by ImageJ. At least three sections near the epicentral part (from dorsal to ventral) of the spinal cord were investigated for each animal.

Anterograde axonal tracing. Axonal regeneration was examined using anterograde tracing (untreated N=7, cells N=7, hydrogel N=10 and implants N=10). Three months after SCI, Tetramethylrhodamine dextran (TMRD; “Fluoro-Ruby”, MW 10,000 kD; Molecular Probes) was injected into the spinal cord at the level of the cervical enlargement, ipsilateral to the lesion (Y. Goldshmit et al., J Neurosci 24, 10064-10073 (2004)). After 14 days, mice were perfused with PBS followed by 4% paraformaldehyde (PFA). Spinal cords were removed and post-fixed for 1 hour in cold 4% PFA followed by 20% sucrose in PBS overnight at 4° C. Longitudinal (horizontal) serial cryostat sections were cut (60 μm) and slides were imaged using fluorescence microscopy (Nikon ECLIPSE NI-E). Labelled axons in the white matter were quantified at 1000 μm, 500 μm, 200 μm rostral to the lesion site and 100 μm caudal to the lesion site at 400×. Photomontage of the regenerating axons was taken using fluorescence microscopy.

Catwalk gait analysis: Gait measurements were collected using the CatWalk XT system (Noldus Information Technology, The Netherlands). Data were transmitted to a computer and analyzed with the CatWalk XT® software (version 10.6, Noldus). Each mouse was located on one side of the walkway and had to complete 3 compliant runs (variation <60%; time <5 sec) to the other side. The coordination (regularity index) and the ability of the mouse to put pressure on the injured paw (left hind max intensity mean) were tested. The parameters were calculated for every run and the results were averaged for every time point per animal.

Grid walk: The mice were tested walking through a horizontal grid (1.2×1.2-cm grid spaces, 35× 45-cm total area), one week prior to the treatment and on weeks 1, 2, 4, 6, 8 post treatment. Each mouse was allowed to walk freely on the grid for 3 minutes. When the left hindlimb paw protruded entirely through the grid with all toes and heel, it was counted as a misstep. The number of missteps and total number of steps taken with the left hindlimb were both counted. The results were expressed as a percentage of correct steps on left hind paw (Y. Goldshmit, Journal of Neurosurgery: Spine SPI 33, 692-704 (2020)).

Magnetic resonance imaging (MRI): MRI was performed on weeks 1 and 4 post chronic scar resection by a Bruker Biospec 7T/30 Scanner equipped with a 660 mT/m gradient unit, using a cross coil configuration of 86 mm transmissive volume coil and 10 mm loop coil as a receiver. For untreated and cells groups N=4, for hydrogel and implants groups N=5. Animals were under 1-2% isoflurane in O₂ anesthesia on a heating pad, with breathing monitored and body temperature maintained at 37° C.

MRI protocol included the following methods: T2 weighted (T2w) images that were acquired using the rapid acquisition with relaxation enhancement (RARE) sequence and Diffusion Tensor Imaging (DTI) acquisition with a Diffusion-Weighted Spin-Echo Echo-Planar-Imaging pulse sequence (DW-SE-EPI). T2w acquisition was performed with the following parameters: TR=8000 ms; effective TE: 30 ms, RARE factor 12 with 3 repetitions. 32 axial slices, 0.45 mm thick (no gaps), with in-plane resolution of 0.15 mm² covering the entire cerebrum, for 4 min. For DTI, performed for 5.5 min, the following parameters were used: TR/TE=2500/19.2 ms, Δ/δ=10/2.5 ms, 2 EPI segment, 30 gradient directions with b-value at 1000 s/mm2 and three B0 images, 30 axial slices, 0.45 mm thick (no gaps), in-plane resolution was 0.30 mm². The total MRI protocol acquisition took ˜20 min. ExploreDTI software was used for DTI calculations and fiber tracking. The eigen-components decomposed from the tensors were used for calculating fractional anisotropy maps. Regions of interest of the spinal cord were manually segmented in each slice. Fiber tracking was employed for tract orientation with angle <30° and FA<0.15 and resolution of 2 2 2.

Statistical analysis: All statistical analyses were performed using GraphPad Prism 8.00 (GraphPad Software, Inc., USA). Data are shown as mean±SEM (Standard Error of the Mean). Data were analyzed using Student's t-test or one-way analysis of variance (ANOVA) followed by Tukey's post-hoc test. The values were considered significantly different at p<0.05.

Rheological properties: Rheological measurements (n=3; individual samples) were taken using Discovery HR-3 hybrid Rheometer (TA Instruments, DE) with 8 mm diameter parallel plate geometry with a peltier plate to maintain the sample temperature. The samples were loaded at a temperature of 4° C., which was then raised to 37° C. to induce gelation; during which the oscillatory moduli of samples were monitored at a fixed frequency of 1 Hz and a strain of 1%. After gelation the same samples were monitored using a frequency sweep (0.1-10 Hz) under 1% strain. Cellular constructs were crosslinked prior to the rheology and were tested at different frequencies as mentioned above.

Scanning electron microscopy: To prepare the samples (either cellular or acellular), the constructs were fixed with 2.5% glutaraldehyde (16-20 hr at 4° C.), followed by graded series of ethanol-water solutions for dehydration (25-100%). All samples were critical point dried, mounted onto aluminum stubs with conductive paint and sputter-coated with gold in a Polaron E 5100 coating apparatus (Quorum technologies, Lewis, UK). Samples were observed under JCM-6000PLUS NeoScope Benchtop (JEOL USA Inc., Peabody, MA).

Flow cytometry: For flow cytometry analysis, cells were isolated from implants using up to six cycles (30 min each) of enzyme digestion with collagenase type II (95 U/mL; Worthington, Lakewood, NJ) and pancreatin (0.6 mg/mL; Sigma-Aldrich) in Dulbecco's modified Eagle Medium (DMEM, CaCl₂)·2H₂O (1.8 mM), KCl (5.36 mM), MgSO₄·7H₂O (0.81 mM), NaCl (0.1 M), NaHCO₃ (0.44 mM), NaH₂PO₄ (0.9 mM)). After each round of digestion cells were centrifuged (120 g, 5 min) and re-suspended in DMEM/D12 and kept on ice. Cells were washed in PBS and then treated with Accutase (Stemcell Technologies, Vancouver. Canada) for 5 min at 37° C. following mechanical trituration to assure dissociation to single cells.

For membrane proteins, cells were stained with conjugated antibody or isotype control for 30 min at RT.

For intracellular proteins, cells were fixed with 4% formaldehyde, washed with PBS, permeabilized with triton 0.1% and incubated with primary and thereafter secondary antibodies for 30 min each, on ice. Cells were analyzed and data analysis was performed using CytoFlex 4 flow cytometer (Beckman Coulter, USA). Positive populations were gated according to unstained cells and appropriate isotype control. At least 3 biological replicates were analyzed.

### Antibody List

Primary antibodies: OCT4 (ab27985, 1:100), Ki67 (ab16667, 1:250), TUJ1 (ab7751/ab18207, 1:500), MAP2 (ab5392, 1:1000), NFM (ab24574, 1:1000), SYP (ab32127, 1:500), Iba1 (ab178846, 1:400); Nestin (ab134017; 1:2000); netrin1 (ab37390; 1:100), slit1 (ab115892; 1:100) and Cytopainter red (ab138893) have been acquired from Abcam (Cambridge, MA). GFAP (DAKO Z0334, 1:1000); NeuN (MAB377; 1:200; Millipore); Collagen I (MA1-26771, 1:2000), and TMRD (dextran, tetramethylrhodamine, D1817, 10000 MW) have been acquired from Invitrogen. HB9 (81.5C10, 1:100) was purchased from DSHB.

Secondary antibodies: Alexa Fluor 488 (1:250; 111-545-003; Jackson); Alexa Fluor 555 (1:500; ab150118, Abcam), Alexa Fluor 647 (1:500; ab150135/ab150175, Abcam). Nuclei were visualized with Hoechst33258 (5 μg/mL).

Conjugated antibodies: TRA-1-60-PE (1:100; 130-122-921, Miltenyi, Germany); SSEA-4 (1:100; 130-122-918, Miltenyi).

### Results

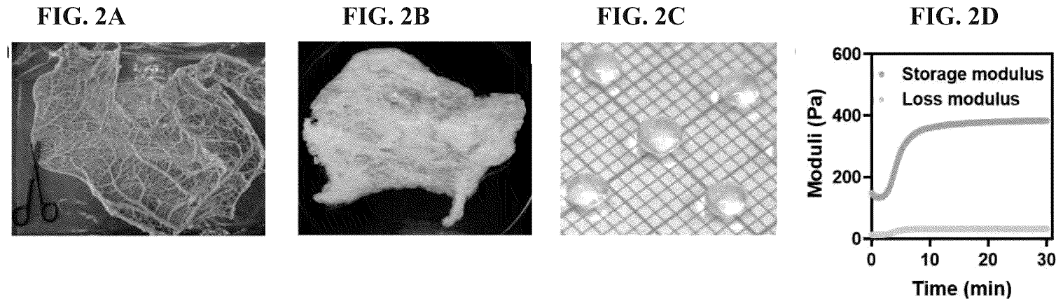

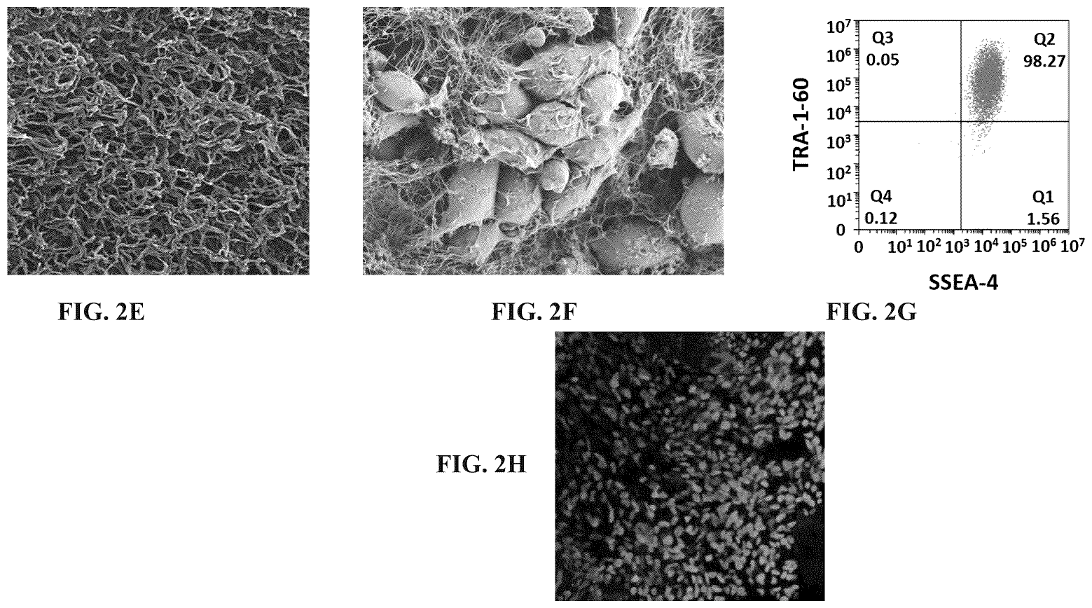

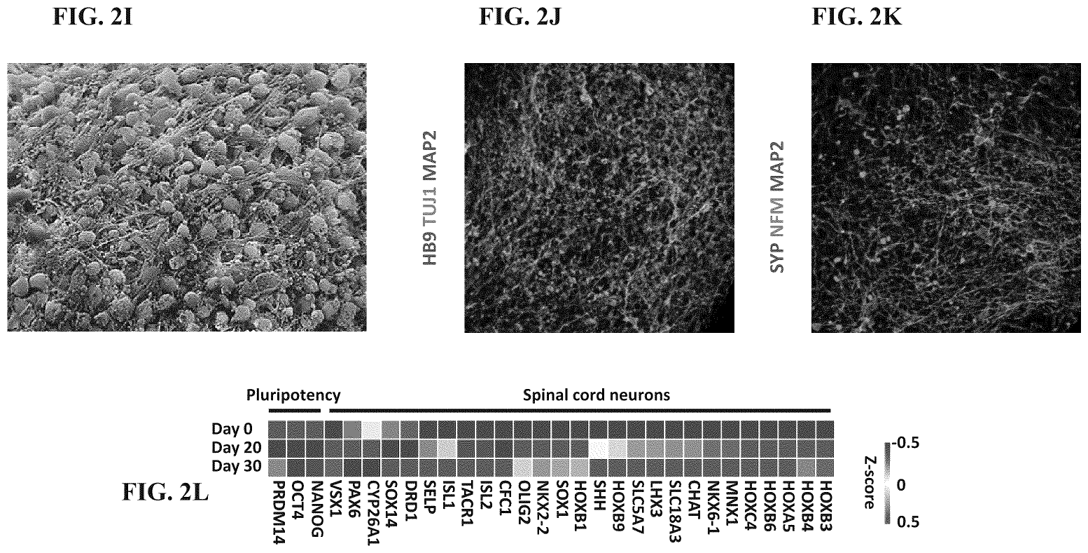

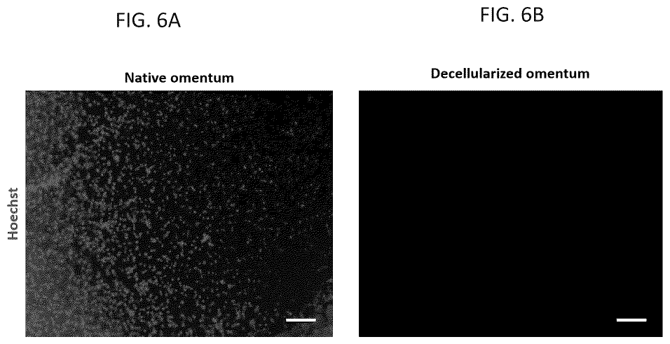

Porcine omental tissue was decellularized while preserving the ECM (FIGS. 2A and 2B and FIGS. 6A-B). The omentum is a fatty tissue enriched with blood vessels and sulfated glycosaminoglycans, and its ECM serves as a depot for stem cells in the body. The decellularized tissue was further processed into a thermoresponsive hydrogel (FIG. 2C), which showed weak mechanical properties at room temperature, and physically crosslinked under physiological conditions (FIGS. 2C and 2D).

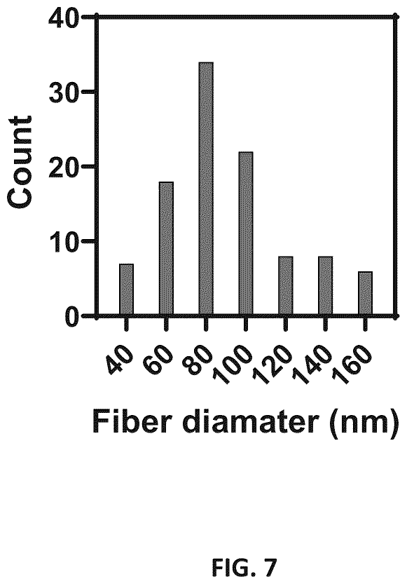

During natural embryonic development, at the blastocyst stage, the fibers of the ECM create niches, which support stem cell renewal, differentiation and morphogenesis. Scanning electron microscopy (SEM) images of the hydrogel revealed its fibrous structure, with an average fiber diameter of 91.7±33 nm (FIG. 2E and FIG. 7). During embryonic development, pluripotent stem cells proliferate in a confined microenvironment prior to differentiation. To mimic this physiological process, human iPSCs colonies were mixed within an omentum hydrogel at a low concentration (FIG. 2F). Particles were generated, as described in the materials and method section. The cells expressed high pluripotency (TRA-1-60, SSEA4, OCT4) and proliferation (KI67) markers within the hydrogel (FIGS. 2G and 2H), and were allowed to proliferate and fill its volume. Then, to mimic the physiological process of neurogenesis, the iPSCs implants (i.e. particles) were subjected to a 30-day differentiation protocol within the 3D microenvironment. On day 30, the cells formed a high-density 3D network within the entire particle (FIG. 2I), expressing general early and late neuronal markers, such as TUJ1 and MAP2, as well as the specific motor neuron marker HB9 (FIG. 2J). Synapses and dendrites, as well as neurofilament formation within the implants, indicated the maturation of neural tissue (FIG. 2K). Flow cytometry analyses indicated that more than 85% of the cells expressed the neuronal marker TUJ1, and more than 60% were also positive for HB9. Moreover, RNA sequencing at 3 different time points along the differentiation process (day 0, day 20 and day 30) revealed downregulation of pluripotency-associated genes, and upregulation of neuronal and specifically spinal cord motor neuron genes (FIG. 2L). Multiple synergistic genes associated with the functions of neuronal activity and maturation were significantly enriched.

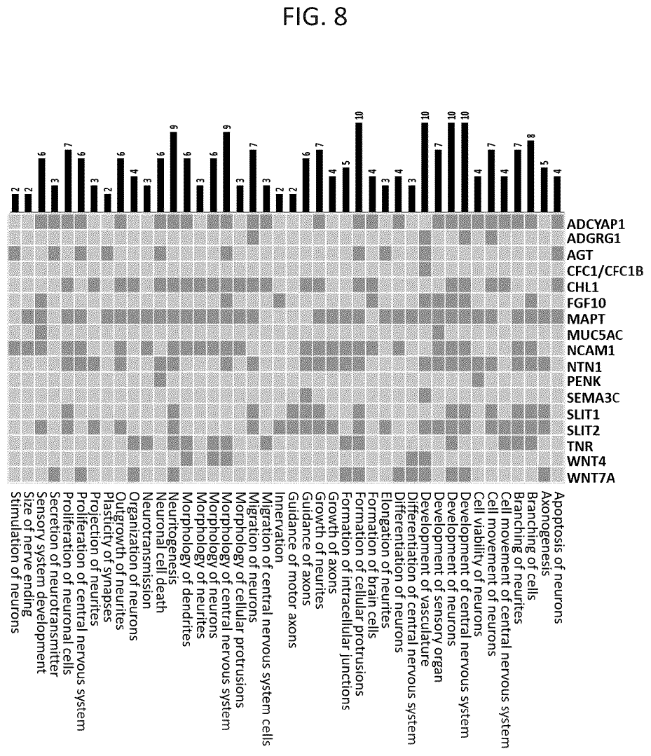

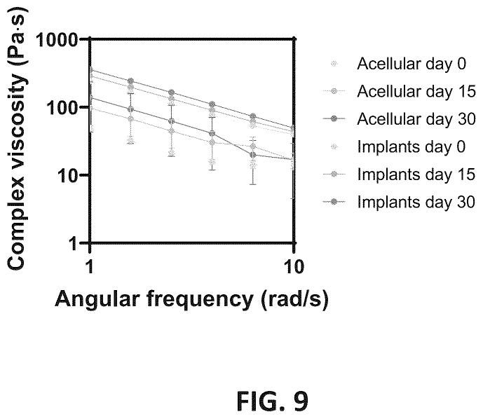

During embryonic development, the dynamic ECM plays a pivotal role in maintaining tissue-specific functions. In addition to the initial matrix proteins, which were provided by the fabricated hydrogel throughout the spinal cord differentiation and maturation, the cells secreted additional specific ECM components and soluble factors, which interacted with the existing matrix. This remodeling of the ECM composition during development and differentiation provides a new microenvironment, which is essential to support cell migration and promote axonal growth/guidance and synaptogenesis. To investigate the new extracellular microenvironment generated during the differentiation process, neuronal ECM-associated genes were studied. Out of the 500 most elevated genes, the cells expressed 17 genes related to production of essential ECM proteins that were not expressed by the undifferentiated cells (FIG. 3A). Further analysis revealed their involvement in enriched functions that are associated with neuronal tissue formation and function, including neuronal development, guidance of axons, neuritogenesis and branching, neuronal migration and neurotransmission (FIG. 3B and FIG. 8). These functions are essential for appropriate formation of the spinal cord during embryonic development²³. Successful mimicking of these capabilities suggests that the supplied microenvironment and the cells mutually affect each other to generate the appropriate 3D neuronal network. While on 2D surfaces most of the soluble proteins are secreted to the medium, in the hydrogel they are spatiotemporally confined within the 3D microenvironment, allowing them to interact with the initial ECM and to affect the encapsulated cells. To assess protein secretion and accumulation within the 3D microenvironment, cells differentiated on MATRIGEL™, undifferentiated implants and 30-day implants were stained for the representative ECM proteins SLIT1 and NTN1, which play a key role in axon guidance and are extremely important for spinal cord positioning. SLIT1 proteins prevent migration of the motor neurons towards the ventral floorplate thereby allowing them to stay in their correct columns. Netrins, on the other hand, are part of the larger laminin gene family and play an important role in guiding axons to the midline. As shown, while the proteins were not detected within the day 0 implants nor on 2D MATRIGEL™ surfaces, they were highly expressed in cells within the hydrogel (FIGS. 3C-E). Synthesis and presentation of ECM proteins affect the function of the developing tissue. However, their accumulation within the 3D hydrogel also altered the microenvironment's mechanical properties. While the complex viscosity of acellular implants was not changed over time, the change in ECM protein type and amount significantly altered its biochemical content, increasing the complex viscosity (FIG. 3F and FIG. 9).

To assess the ability of the implants to interact with their surrounding environment, isolated implants were seeded on a thin layer of MATRIGEL™ and neurite outgrowth was demonstrated (FIG. 3G). Furthermore, when several implants were positioned at a distance of ˜1 mm from each other, a branched network was formed between them within 3 days (FIG. 3H). Such an interaction between implants, or between implants and the healthy part of the tissue, is extremely important for efficient engraftment and for initiating regenerative processes in any neural tissue (Kawabata et al stem cell rep 2016). After confirming the formation of the 3D network, dendrites and synapses, the implants' neural electrical activity was monitored using calcium imaging. KCl is known to reliably depolarize neurons leading to calcium ions flow into the cells. As shown, KCl stimulation revealed a significant increase in fluorescence, indicating the chemically-induced effect (FIG. 3I). Furthermore, the excitatory neurotransmitter glutamate was able to induce a significant increase in calcium release (FIG. 3J).

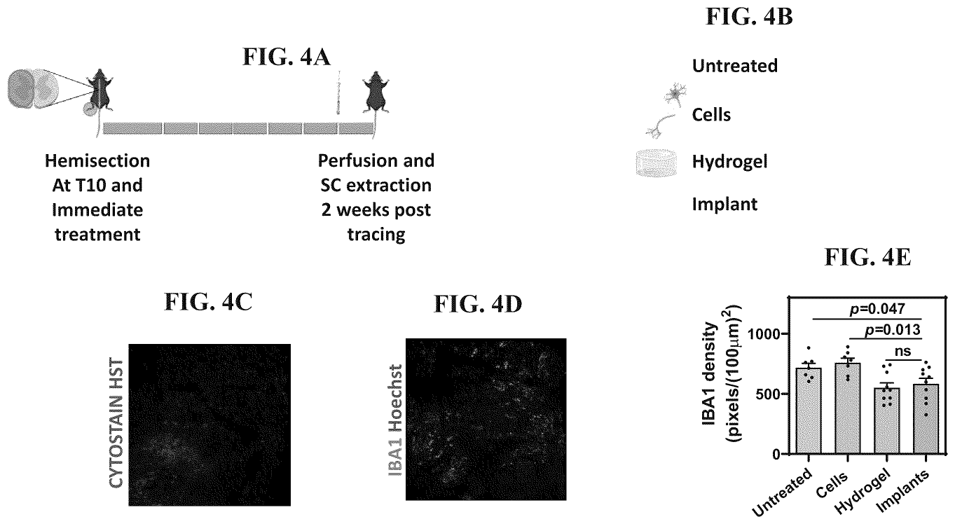

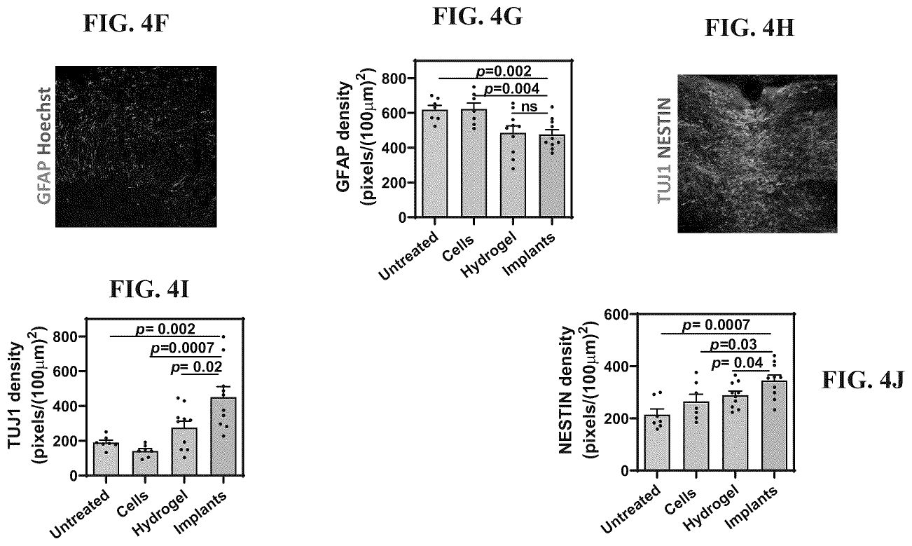

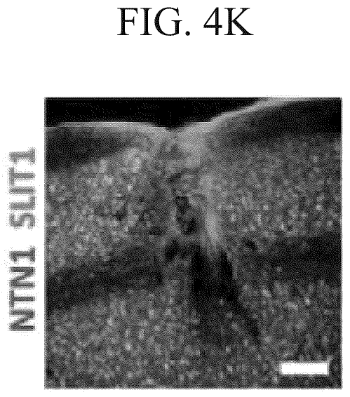

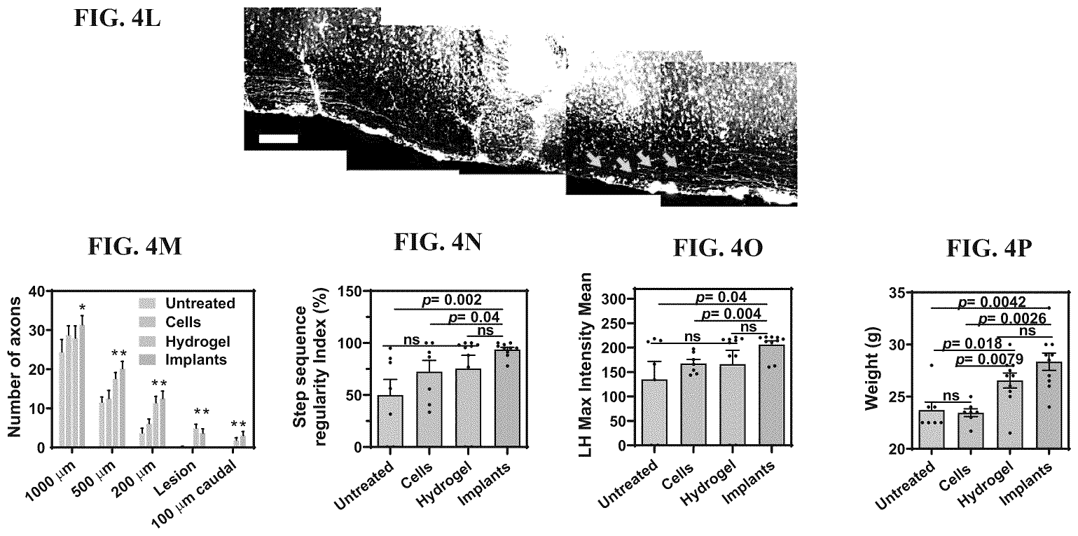

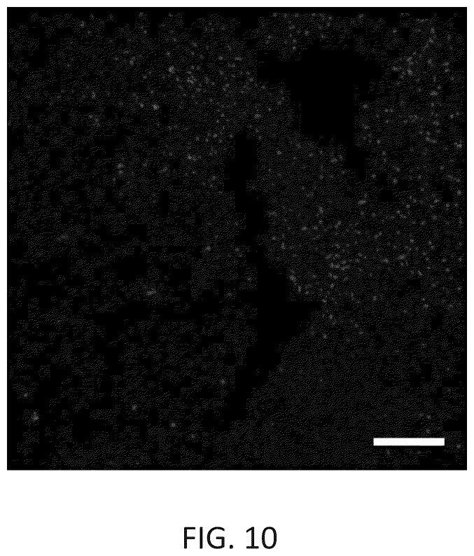

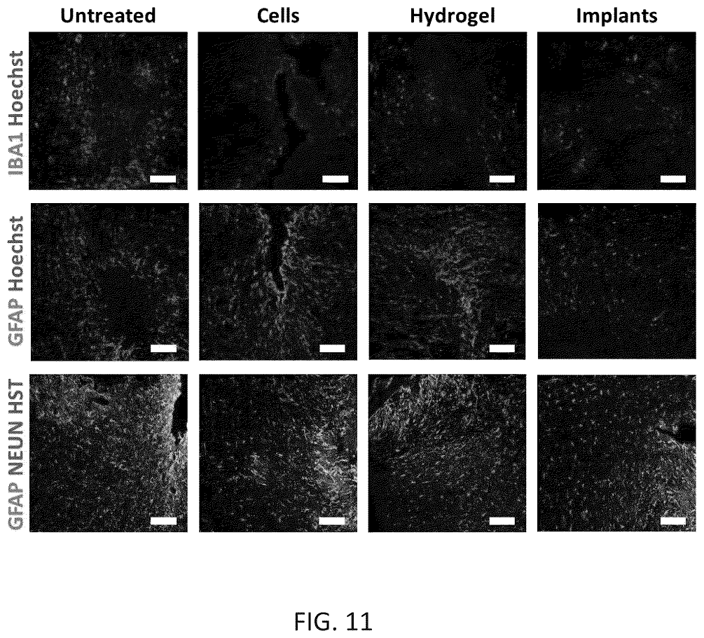

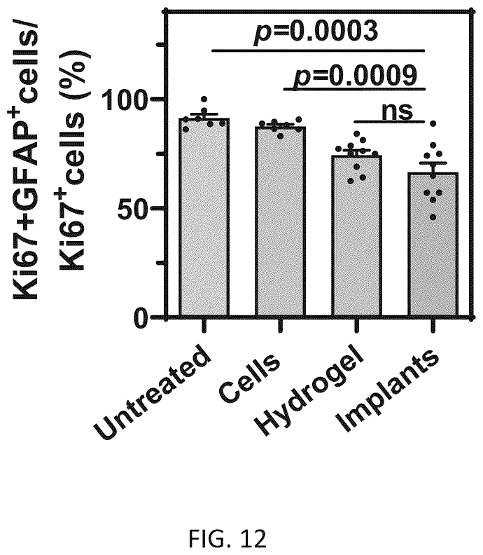

After efficiently mimicking the embryonic spinal cord development and engineering functional tissue implants, their therapeutic potential was evaluated. Initially, as a proof of concept, an acute injury model in mice was chosen. Here, a complete left side hemisection at T10 was performed with the right side of the spinal cord remaining intact (FIG. 4A). Then, saline (untreated), cell suspension in saline (cells), hydrogel without cells or the full implants (FIG. 4B) were immediately inserted into the injury site and their ability to reduce inflammation and glial scar formation, promote neuroprotection and axonal regeneration and improve mice locomotion, were assessed. To compare between the engraftment of dissociated cells and that of the full implant, the cells were first pre-stained with a fluorescent dye. One week after treatment, the cells within the implant group were clearly detected at the lesion site, while cells that were applied in suspension were hardly observed, emphasizing the importance of a supporting microenvironment (FIG. 4C and FIG. 10). After disruption of the blood-spinal cord barrier and hemorrhage, a secondary damage occurs and immune cells from the periphery or microglia from within the tissue migrate towards the injury site. This pro-inflammatory environment results in additional neuronal death and massive accumulation of reactive astrocytes forming a glial scar. This process prevents further damage expansion; however, it inhibits spontaneous regeneration. To assess the effect of the implants on the inflammatory cell population within the injury site, the spinal cord was extracted on day 7, sectioned and stained for microrglia (Iba1) and astrocytes (GFAP) markers. As shown, both the hydrogel and the implants significantly reduced the accumulation of both cell types (FIGS. 4C-G and FIG. 11). Moreover, the astrocytes detected within these groups were less reactive, as they also expressed Ki67 (FIG. 12).

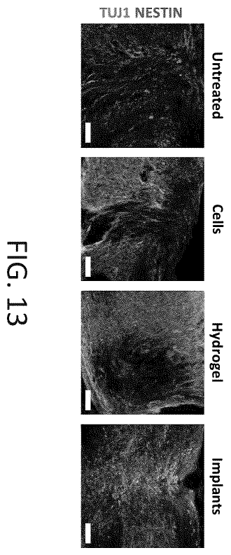

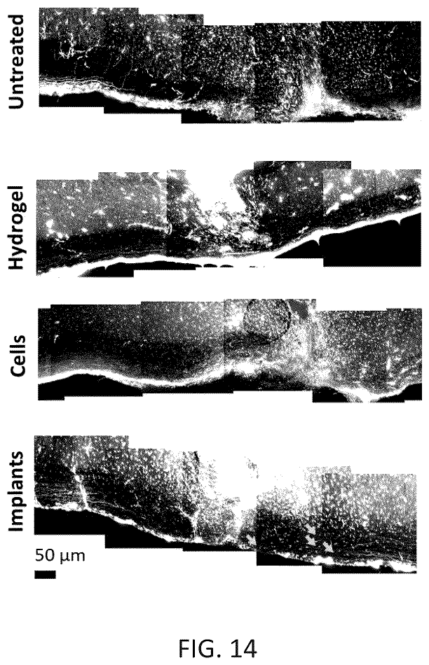

The lower level of inflammation observed on day 7 was translated to a more permissive environment, which resulted in significantly higher numbers of neurons (TUJ1) and neural stem cells (NESTIN) detected on week 12 in animals treated with the implants (FIGS. 4H-J and FIG. 13). The latter are progenitor cells with a capacity to differentiate to neurons or glial cells. Overall the cells at the lesion site were organized in the direction of the spinal cord tracts, bridging over the injury (FIG. 4H). This may be attributed to the high levels of guidance molecules observed within the surrounding microenvironment (FIG. 14). Overall, such an organization of cells and essential ECM proteins may promote rewiring and regeneration across the injured spinal cord.

To evaluate the potential of the implant to transfer a signal through the lesion site, mice were injected with an anterograde tracer molecule (TMRD) at the cervical level ipsilateral to the injury. The animals were kept for an additional 2 weeks to allow the tracer to migrate downstream through active neuronal axons. As shown, implant-treated mice had a significantly higher number of axons that had reached and passed the lesion site, allowing regrowth through the scar, whereas lower numbers of axons were detected in hydrogel-treated mice, and none were observed in the other treatments (FIGS. 4K and 4L and FIG. 15).

The presence of functional axons along the spinal cord tracts is essential for proper motor function. Therefore, the present inventors next sought to assess the ability of the implants to improve the locomotion of the treated mice by Catwalk gait analysis. As shown, all animals regained partial motor function, probably due to their ability to use the central pattern generator. However, the recovery was significantly improved in animals that were treated with the implants (FIGS. 4M and 4N). Compared to the untreated group, only animals treated with the implant showed a significantly better coordination, as judged by the higher regularity index (FIG. 4M). Similarly, left hind maximal intensity, which indicates the ability of the animal to apply pressure to the injured foot, was significantly higher only in animals treated with the implant (FIG. 4N). These improved behavioral parameters may be attributed to the synergistic effect of decreased inflammation and the presence of neurons in the lesion site, which are essential for regeneration. This recovery was also translated to a significantly higher gain in weight (FIG. 4O), showing another aspect of the overall improved condition.

Having demonstrated the ability of the implants to recover the injured spinal cord in the acute phase, their ability to regenerate the tissue in a more clinically relevant model was subsequently assessed. Immediately after initial trauma, a secondary cascade of events, including bleeding and edema, takes place. Therefore, the present inventors sought to assess the potential of the implant to treat the injured spinal cord once the damage has reached the chronic phase, where the scar is fully developed, and spontaneous behavioral recovery has reached its plateau.

For this purpose, a complete hemisection was performed, as described for the acute phase. Six weeks after the initial SCI, the scar was ablated from the spinal cord, and the same treatments were applied in the cavity (FIG. 4B). Subsequently, the structural, biochemical, cellular and behavioral parameters were assessed (FIG. 5A).

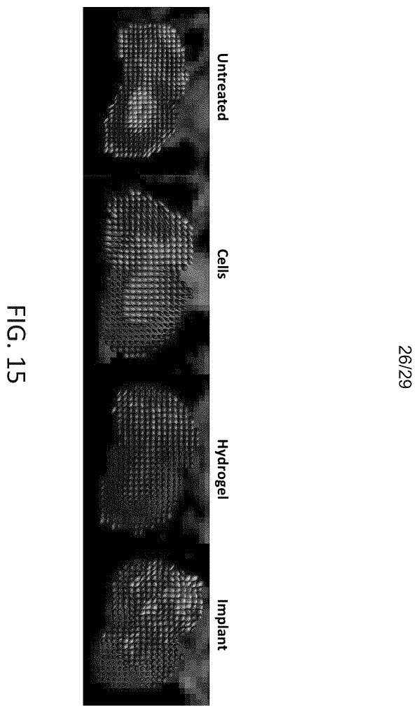

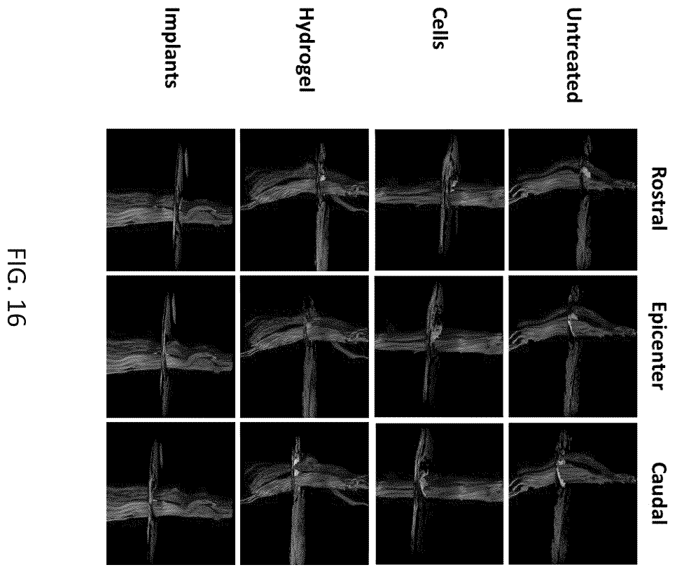

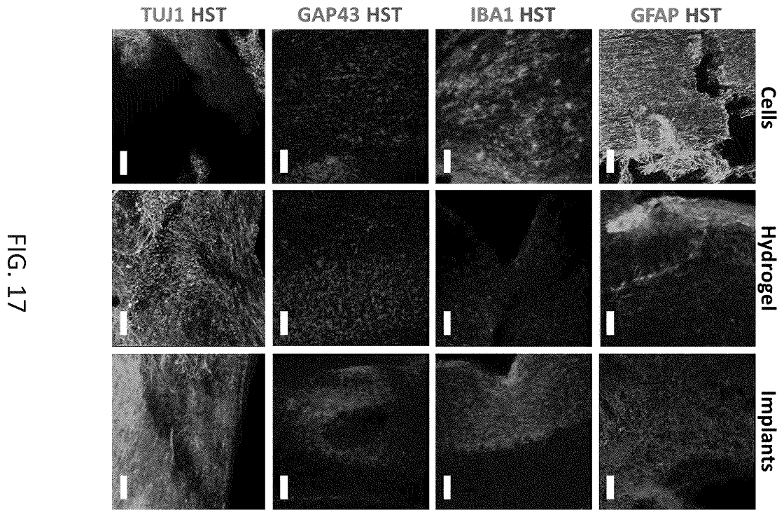

As shown, the complete hemisection could be easily detected by T2-weighted MRI prior to scar resection (FIG. 5B). To visualize the repair process of the spinal cord tracts, MRI with diffusion tensor imaging (DTI) was performed at 1- and 4-weeks post treatment. Diffusion anisotropy, which provides valuable information on the white matter of the spinal cord, is determined both by the axonal structural orientation and the state of myelin. The diffusion tensor in each voxel of an MRI image is represented by the principal eigenvector, indicating the fibers main orientation within the voxel. As shown, one week after scar resection, the main diffusion orientation was random, rather than aligned along the spinal cord axis (FIG. 16), which may indicate a severe injury. However, at this stage of injury, it is likely that some of the detected damage was caused by edema after the scar resection, which could be completely or partially reversible. Analysis at 4-weeks after treatment revealed a major improvement in animals treated with the implants, as judged by the main diffusion orientation (indicated by their blue color) (FIG. 5C). This improvement may be attributed to the integration of the implant and its ability to bridge between healthy axons above and below the lesion site. Streamline tractography was then used to visualize white matter fibers, their structural integrity, and the damage to the fiber bundle. Reconstructed neuronal tracts showed pathological changes in the spinal cord at the lesion site for all animals (FIG. 5D). However, tractography of untreated animals or animals treated with cells in suspension revealed higher displacement, deformation and disruption in lesions. In these animals, only a few tissues or axons passing across the injured site were revealed, along with gradual Wallerian degeneration above the level of scar resection. In contrast, animals treated with the implants exhibited better preservation or restoration of the injured tracts, as judged by the number of tracts passing the lesion site (FIG. 5D, FIG. 17). The amount of ipsilateral nerve fibers crossing the injury site was quantified and compared to the fibers on the same levels on the healthy side. As shown, implants-treated mice had significantly higher survival and/or regrowth of axons through the lesion, compared to untreated and cell-treated animals (FIG. 5E). The present inventors next analyzed the fractional anisotropy, which depends on the water diffusivity in the extracellular space along the axons. Compared to all other treatments, animals treated with implants revealed significantly higher values at the lesion site (FIG. 5F), indicating stronger anisotropy and higher number of complete nerve fibers.

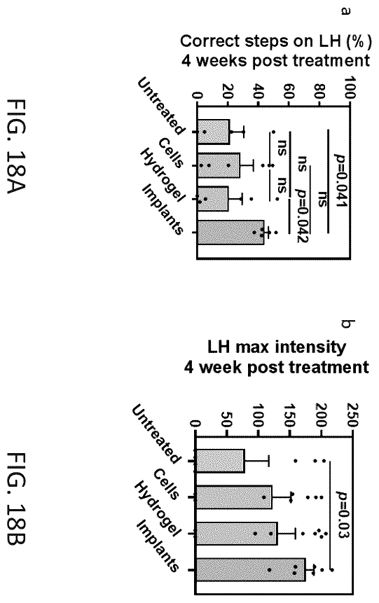

The cellular content at the injury site was analyzed after a longer recovery period. The extensive damage caused by the removal of the scar tissue, with no further insertion of a supporting material, left a substantial cavity in several of the untreated animals. These spinal cords could not be extracted and processed in one piece, preventing quantification of the cellular content at the injury site of these animals and reliable analysis of this group. Comparison between the cells, hydrogel and implant treatments revealed a significantly reduced chronic inflammation in animals treated with the implants, as evident by the lower density of reactive astrocytes (FIG. 5G and FIGS. 18A-B). Microglia was also significantly reduced by both the hydrogel and implant treatments compared to the cells only group (FIG. 5H and FIGS. 18A-B). As shown, significantly higher numbers of neurons were found both in hydrogel and implants-treated mice (FIG. 5I and FIGS. 18A-B). However, higher expression of GAP43, a marker associated with sprouting of axons in development and regeneration of the spinal cord, was only detected in the implant-treated animals (FIG. 5J and FIGS. 18A-B). This may suggest active axonal sprouting at the lesion site, which may gap the healthy axons on both sides of the lesion.

The potential of the implants to promote functional recovery was validated by sensorimotor function. During the experiment, mice were subjected to behavioral studies including Catwalk gate analysis and grid walk test. The coordination of mice, represented by step sequence regularity index, improved over time and reached its full potential 6 weeks after treatment (FIG. 5K). Furthermore, higher pressure placed on the injured foot was detected already at one-week post implantation in implant-treated animals and was maintained throughout the experiment (FIG. 5L). Finally, motoric and sensory recovery was observed in these animals, as indicated by less missed steps on the injured foot in grid walk analysis (FIG. 5M). The significant recovery detected in the sensorimotor of the implant-treated animals is in agreement with the results of the DTI, revealing intact fibers crossing the injury site (FIG. 5D).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety

What is claimed is:

1. A composition comprising a plurality of fibrous particles fabricated from decellularized omentum, said fibrous particles being between 750 microns-3 mm in diameter, wherein said fibrous particles comprise a network of mature neurons.

2. The composition of claim 1, wherein said fibrous particles are essentially spherical.

3. The composition of claim 1, wherein said mature neurons comprise motor neurons.

4. The composition of claim 3, wherein more than 50% of the motor neurons express Neuron-specific class III beta-tubulin (TUJ1), as measured by flow cytometry.

5. The composition of claim 3, wherein more than 50% of the cells express Motor neuron and pancreas homeobox 1 (MNX1), as measured by flow cytometry.

6. The composition of claim 1, wherein fibers of said fibrous particles have an average diameter between 50-200 nm in diameter.

7. The composition of claim 1, wherein said omentum comprises human omentum.

8. A method of treating a chronic spinal cord injury of a subject comprising transplanting the composition of claim 1 into the subject at the site of injury, at least three months following the spinal cord injury, thereby treating the spinal cord injury.

9. The method of claim 8, wherein said transplanting is affected at least six months following the spinal cord injury.

10. The method of claim 8, further comprising removing scar tissue at the site of injury from said subject prior to said transplanting.

11. The method of claim 10, wherein the composition comprises a pharmaceutically acceptable carrier at the time of transplanting and the method further comprises removing at least a portion of said carrier from said site of injury following said transplanting.

12. The method of claim 8, wherein said transplanting is affected using a syringe.

13. The method of claim 12, wherein an inner diameter of said syringe is between 1-5 mm.

14. An article of manufacture comprising:
(i) the composition of claim 1; and
(ii) a device for delivering the composition into a spinal cord of a subject.

15. The article of manufacture of claim 14, wherein said device is a syringe, and an inner diameter of said syringe is between 1-5 mm.

16. A method of generating the composition of claim 1, comprising:
(a) generating particles of decellularized omentum which comprise pluripotent stem cells, said particles having a diameter between 750 microns-3 mm;
(b) contacting said particles with at least one neuronal differentiation agent; and
(b) culturing said particles in the presence of said at least one neuronal differentiating agent under conditions that promote generation of a neuronal network of mature neurons in said particles, thereby generating the composition of claim 1.

17. The method of claim 16, wherein said pluripotent stem cells are induced pluripotent stem cells which are optionally reprogrammed from omental stromal cells.

18. The method of claim 16, wherein said neuronal differentiation agent is selected from the group consisting of a Transforming Growth Factor Beta Receptor 1 (ALK-5) inhibitor, morphogenic protein 4 (BMP4) inhibitor, retinoic acid, bone derived neurotrophic factor (BDNF), ascorbic acid and purmorphamine.

19. The method of claim 18, wherein said mature neurons comprise motor neurons.

20. The method of claim 19, wherein:
(i) more than 50% of the motor neurons express Neuron-specific class III beta-tubulin (TUJ1), as measured by flow cytometry; and/or
(ii) more than 50% of the cells express Motor neuron and pancreas homeobox 1 (MNX1), as measured by flow cytometry.
